(19) United States
(12) Patent Application Publication
(10) Pub. No.: US 2010/0120126 A1
(43) Pub. Date: May 13, 2010

(54) MICROORGANISMS PRODUCING DIPEPTIDES AND PROCESS FOR PRODUCING DIPEPTIDE USING THE MICROORGANISMS

(75) Inventors: Shin-ichi Hashimoto, Yamaguchi (JP); Kazuhiko Tabata, Tokyo (JP)

Correspondence Address: FITZPATRICK CELLA HARPER & SCINTO, 1290 Avenue of the Americas, NEW YORK, NY, 10104-3800, US

(73) Assignee: KYOWA HAKKO BIO CO., LTD., Tokyo (JP)

(21) Appl. No.: 12/615,337

(22) Filed: Nov. 10, 2009

Related U.S. Application Data
(62) Division of application No. 10/976,042, filed on Oct. 29, 2004, now abandoned.

(30) Foreign Application Priority Data
Nov. 5, 2003 (JP) 2003-375823
Jun. 25, 2004 (JP) 2004-189010

(51) Int. Cl. C12N 1/21

(52) U.S. Cl. 435/252.31; 435/252.3; 435/252.32; 435/252.33

(57) ABSTRACT

Microorganisms in which the activities of one or more kinds of peptidases and one or more kinds of proteins having peptide-transporting activity are reduced or lost and which have the ability to produce a dipeptide. Also, microorganisms in which the activities of three or more kinds of peptidases are reduced or lost and which have the ability to produce a dipeptide, and a process for producing dipeptides using the microorganisms.

This application is a division of Division of application Ser. No. 10/976,042 filed Oct. 29, 2004, which claims priority of Japanese Patent Application Nos. JP 2003-375823 filed Nov. 5, 2003 and JP 2004-189010 filed Jun. 25, 2004.

### BACKGROUND OF THE INVENTION

The present invention relates to a microorganism producing a dipeptide and a process for producing a dipeptide using the microorganism.

Dipeptides are compounds that are important as foods, pharmaceuticals, cosmetics and the like.

Known methods for producing dipeptides include extraction from natural products, chemical synthesis and enzymatic methods. Extraction from natural products can be used only for producing limited kinds of dipeptides, and the productivity is low because the contents of desired dipeptides in natural products are low. In the synthesis of dipeptides by the chemical synthesis methods, operations such as introduction and removal of protective groups for functional groups are necessary, and racemates are also formed. The chemical synthesis methods are thus considered to be disadvantageous in respect of cost and efficiency. They are unfavorable also from the viewpoint of environmental hygiene because of the use of large amounts of organic solvents and the like.

As to the synthesis of dipeptides by the enzymatic methods, the following methods are known: a method utilizing reverse reaction of protease (J. Biol. Chem., 119, 707-720 (1937)); methods utilizing thermostable aminoacyl t-RNA synthetase (Japanese Published Unexamined Patent Application No. 146539/83, Japanese Published Unexamined Patent Application No. 209991/83, Japanese Published Unexamined Patent Application No. 209992/83 and Japanese Published Unexamined Patent Application No. 106298/84); methods utilizing non-ribosomal peptide synthetase (hereinafter referred to as NRPS) (Chem. Biol., 7, 373-384 (2000), FEBS Lett., 498, 42-45 (2001), U.S. Pat. No. 5,795,738 and U.S. Pat. No. 5,652,116); a method utilizing D-Ala-D-Ala ligase (Biochemistry, 35, 10464-10471 (1996)); methods utilizing bacilysin synthetase (J. Ind. Microbiol., 2, 201-208 (1987) and Enzyme. Microbial. Technol., 29, 400-406 (2001)); and a method utilizing L-amino-acid amide hydrolase or proline iminopeptidase (WO03/010307 pamphlet).

However, the method utilizing reverse reaction of protease requires introduction and removal of protective groups for functional groups of amino acids used as substrates, which causes difficulties in raising the efficiency of peptide-forming reaction and in preventing peptide-degrading reaction. The methods utilizing thermostable aminoacyl t-RNA synthetase have the defects that the expression of the enzyme and the prevention of reactions forming by-products are difficult.

On the other hand, the methods utilizing NRPS, D-Ala-D-Ala ligase and bacilysin synthetase do not have the problems described above and are capable of producing dipeptides having specific sequences. However, they are not efficient methods because they involve reactions requiring energy donors such as ATP.

With regard to the method utilizing L-amino-acid amide hydrolase or proline iminopeptidase, it is disclosed that dipeptides can be produced by using a culture of a microorganism producing the enzyme, microorganism cells isolated from the culture or a treated matter of the microorganism cells. However, the amounts of dipeptides produced by this method are not sufficient.

Living cells have metabolic systems in which unnecessary proteins are decomposed to constituent amino acids and the formed amino acids are used for synthesis of necessary proteins. As this function is essential for the survival and proliferation of cells, it is known that many kinds of proteases and peptidases exist in living organisms.

For example, it is known that in Escherichia coli, there exist at least seven kinds of dipeptidases as well as other proteases and peptidases capable of decomposing dipeptides (FEMS Microbial. Rev., 63, 265-276 (1989)). On the chromosomal DNAs of Corynebacterium glutamicum, Bacillus subtilis and Saccharomyces cerevisiae, there exist 25 or more, 14 or more, and 20 or more, respectively, genes identified as peptidase gene.

However, it is not known that introduction of peptidase deficiency into a microorganism producing a dipeptide increases the production of the dipeptide.

It is also known that living cells have plural peptide-incorporating systems. For example, Escherichia coli is reported to have three systems for incorporating dipeptides (Chem. Biol., 5, 489-504 (1998) and Microbiol., 145, 2891-2901 (1999)). Further, it has been confirmed from genomic DNA information that microorganisms belonging to the genera Escherichia, Bacillus, Corynebacterium and Saccharomyces all have three or more kinds of peptide-incorporating systems.

However, it is not known whether dipeptides synthesized in cells are discharged outside the cells or not. Still less is known that the production of a dipeptide by a dipeptide-producing microorganism is increased by introducing deficiency of a protein involved in the peptide incorporation into the microorganism.

An object of the present invention is to provide a microorganism producing a dipeptide and a process for producing a dipeptide using the microorganism.

### SUMMARY OF THE INVENTION

The present invention relates to the following (1) to (21).
(1) A microorganism in which the activities of one or more kinds of peptidases and one or more kinds of proteins having peptide-transporting activity (hereinafter referred to also as peptide-transporting proteins) are reduced or lost and which has the ability to produce a dipeptide.
(2) A microorganism in which the activities of three or more kinds of peptidases are reduced or lost and which has the ability to produce a dipeptide.
(3) The microorganism according to the above (1) or (2), wherein the peptidase is a protein having the amino acid sequence shown in any of SEQ ID NOS: 1 to 4, or a protein having an amino acid sequence which has 80% or more homology to the amino acid sequence shown in any of SEQ ID NOS: 1 to 4 and having peptidase activity.
(4) The microorganism according to the above (1) or (3), wherein the peptide-transporting protein is a protein having the amino acid sequence shown in any of SEQ ID NOS: 5 to 9, or a protein having an amino acid sequence which has 80% or more homology to the amino acid sequence shown in any of SEQ ID NOS: 5 to 9 and having peptide-transporting activity.
(5) The microorganism according to any of the above (1) to (4), wherein the microorganism which has the ability to produce a dipeptide is a microorganism having the ability to produce a protein according to any of [1] to [4] below:
   [1] a protein having the amino acid sequence shown in any of SEQ ID NOS: 19 to 25 and 68;
   [2] a protein consisting of an amino acid sequence wherein one or more amino acid residues are deleted, substituted or added in the amino acid sequence shown in any of SEQ ID NOS: 19 to 25 and 68 and having the activity to synthesize a dipeptide;
   [3] a protein consisting of an amino acid sequence which has 65% or more homology to the amino acid sequence shown in any of SEQ ID NOS: 19 to 25 and 68 and having the activity to synthesize a dipeptide; and
   [4] a protein having an amino acid sequence which has 80% or more homology to the amino acid sequence shown in SEQ ID NO: 33 and having the activity to synthesize a dipeptide.
(6) The microorganism according to any of the above (1) to (4), wherein the microorganism which has the ability to produce a dipeptide is a microorganism carrying DNA according to any of [1] to [4] below:
   [1] DNA encoding the protein according to any of [1] to [4] of the above (5);
   [2] DNA having the nucleotide sequence shown in any of SEQ ID NOS: 26 to 32, 64 and 65;
   [3] DNA which hybridizes with DNA having the nucleotide sequence shown in any of SEQ ID NOS: 26 to 32, 64 and 65 under stringent conditions and which encodes a protein having the activity to synthesize a dipeptide; and
   [4] DNA having a nucleotide sequence which has 80% or more homology to the nucleotide sequence shown in SEQ ID NO: 34 and encoding a protein having the activity to synthesize a dipeptide.
(7) The microorganism according to any of the above (1) to (6), wherein the microorganism which has the ability to produce a dipeptide is a microorganism carrying a recombinant DNA in which the DNA according to any of [1] to [4] of the above (6) is ligated to a vector DNA.
(8) The microorganism according to any of the above (1) to (7), wherein the microorganism is a microorganism belonging to the genus Escherichia, Bacillus, Corynebacterium or Saccharomyces.
(9) The microorganism according to any of the above (1) to (4), wherein the microorganism which has the ability to produce a dipeptide is a microorganism having the ability to form a dipeptide from an L-amino acid ester and an L-amino acid.
(10) The microorganism according to the above (9), wherein the microorganism having the ability to form a dipeptide from an L-amino acid ester and an L-amino acid is a microorganism producing proline iminopeptidase.
(11) The microorganism according to the above (9) or (10), wherein the microorganism having the ability to form a dipeptide from an L-amino acid ester and an L-amino acid is a microorganism carrying a recombinant DNA in which DNA encoding a protein having proline iminopeptidase activity is ligated to a vector DNA.
(12) The microorganism according to any of the above (9) to (11), wherein the microorganism is a microorganism belonging to the genus Escherichia, Bacillus, Corynebacterium or Saccharomyces.
(13) The microorganism according to any of the above (1) to (4), wherein the microorganism is a microorganism having the ability to form a dipeptide from an L-amino acid amide and an L-amino acid.
(14) The microorganism according to the above (13), wherein the microorganism having the ability to form a dipeptide from an L-amino acid amide and an L-amino acid is a microorganism producing a protein having L-amino acid amide hydrolase activity.
(15) The microorganism according to the above (13), wherein the microorganism having the ability to form a dipeptide from an L-amino acid amide and an L-amino acid is a microorganism carrying a recombinant DNA in which DNA encoding a protein having L-amino acid amide hydrolase activity is ligated to a vector DNA.
(16) The microorganism according to any of the above (13) to (15), wherein the microorganism is a microorganism belonging to the genus Escherichia, Bacillus, Corynebacterium or Saccharomyces.
(17) A process for producing a dipeptide, which comprises:
   allowing an enzyme source and one or more kinds of amino acids to be present in an aqueous medium, said enzyme source being a culture of the microorganism according to any of the above (1) to (8) or a treated matter of the culture;
   allowing the dipeptide to form and accumulate in the aqueous medium; and
   recovering the dipeptide from the medium.
(18) The process according to the above (17), wherein the dipeptide is a dipeptide represented by the following formula (I):

R¹-R² (I)
(wherein R¹ and R², which may be the same or different, each represent an amino acid).
(19) A process for producing a dipeptide, which comprises:
   allowing an enzyme source, an L-amino acid ester and an L-amino acid to be present in an aqueous medium, said enzyme source being a culture of the microorganism according to any of the above (9) to (12) or a treated matter of the culture;
   allowing the dipeptide to form and accumulate in the aqueous medium; and
   recovering the dipeptide from the medium.
(20) A process for producing a dipeptide, which comprises:
   allowing an enzyme source, an L-amino acid amide and an L-amino acid to be present in an aqueous medium, said enzyme source being a culture of the microorganism according to any of the above (13) to (16) or a treated matter of the culture;
   allowing the dipeptide to form and accumulate in the aqueous medium; and
   recovering the dipeptide from the medium.
(21) The process according to any of the above (17) to (20), wherein the treated matter of the culture is concentrated culture, dried culture, cells obtained by centrifuging the culture, or a product obtained by subjecting the cells to drying, freeze-drying, treatment with a surfactant, ultrasonication, mechanical friction, treatment with a solvent, enzymatic treatment or immobilization.

In accordance with the present invention, there are provided a microorganism producing a dipeptide and a process for producing a dipeptide using the microorganism.

### BRIEF DESCRIPTION OF THE DRAWINGS

FIG. 1 shows the steps for constructing His-tagged ywfE gene expression vector pQE60ywfE.

FIG. 2 shows the steps for constructing ywfE gene expression-enhanced vector pPE56.

### EXPLANATION OF SYMBOLS

PT5: T5 promoter
Ptrp: Tryptophan promoter

### DETAILED DESCRIPTION OF THE INVENTION

The present invention is described in detail below.

### 1. Microorganisms of the Present Invention

The microorganisms of the present invention are microorganisms in which the activities of one or more kinds of peptidases and one or more kinds of proteins having peptide-transporting activity (hereinafter referred to as peptide-transporting proteins) are reduced or lost and which have the ability to produce a dipeptide, or microorganisms in which the activities of three or more kinds of peptidases are reduced or lost and which have the ability to produce a dipeptide.

The microorganisms in which the activities of one or more kinds of peptidases and one or more kinds of peptide-transporting proteins are reduced or lost include microorganisms in which the activities of one or more arbitrary kinds of peptidases and one or more arbitrary kinds of peptide-transporting proteins are reduced or lost provided that the microorganisms can normally grow, specifically, microorganisms in which the activities of preferably one to nine kinds, more preferably one to seven kinds, further preferably one to four kinds of peptidases and preferably one to five kinds, more preferably one to three kinds, further preferably one or two kinds, particularly preferably one kind of peptide-transporting protein are reduced or lost.

Examples of such microorganisms are microorganisms in which the activities of one or more kinds of peptidases and one or more kinds of peptide-transporting proteins are reduced or lost because the nucleotide sequences of one or more kinds of genes encoding peptidases (hereinafter referred to as peptidase genes) and one or more kinds of genes encoding peptide-transporting proteins (hereinafter referred to as peptide-transporting protein genes) among the peptidase genes and peptide-transporting protein genes existing on the genomic DNA of the microorganisms are entirely or partially deleted or said nucleotide sequences contain nucleotide substitutions or additions.

The expression “the activity of peptidase is reduced” means that the peptide-degrading activity is reduced compared with peptidase having none of the above deletions, substitutions and additions of nucleotides.

The peptide-degrading activity of a microorganism can be measured by allowing a peptide as a substrate and microorganism cells to be present in an aqueous medium, thereby performing peptide-degrading reaction, and then determining the amount of the remaining peptide by a known method, e.g., HPLC analysis.

The above peptidases may be any proteins having peptide-degrading activity. Preferred are proteins having high dipeptide-hydrolyzing activity. More preferred are dipeptidases.

Examples of peptidases include: those existing in Escherichia coli such as PepA having the amino acid sequence shown in SEQ ID NO: 1, PepB having the amino acid sequence shown in SEQ ID NO: 2, PepD having the amino acid sequence shown in SEQ ID NO: 3, PepN having the amino acid sequence shown in SEQ ID NO: 4, PepP [GenBank accession No. (hereinafter abbreviated as Genbank) AAC75946], PepQ (GenBank AAC76850), PepE (GenBank AAC76991), PepT (GenBank AAC74211), Dcp (GenBank AAC74611) and IadA (GenBank AAC77284); those existing in Bacillus subtilis such as AmpS (GenBank AF012285), PepT (GenBank X99339), YbaC (GenBank Z99104), YcdD (GenBank Z99105), YjbG (GenBank Z99110), YkvY (GenBank Z99111), YqjE (GenBank Z99116) and YwaD (GenBank Z99123); those existing in Corynebacterium glutamicum such as proteins having the amino acid sequences represented by BAB97732, BAB97858, BAB98080, BAB98880, BAB98892, BAB99013, BAB99598 and BAB99819 (registration Nos. of DNA Data Bank of Japan); and those existing in Saccharomyces cerevisiae such as OCT1 (GenBank NC_{—}001143), SPC2 (GenBank NC_{—}003143), SPY2 [Saccharomyces genome database accession No. L0002875] and YIM1 (GenBank NC_{—}001145). Examples of dipeptidases include PepA, PepB, PepD and PepN having the amino acid sequences shown in SEQ ID NOS: 1 to 4, PepQ, PepE and IadA. Proteins having amino acid sequences which have 80% or more, preferably 90% or more, more preferably 95% or more homology to the amino acid sequence shown in any of SEQ ID NOS: 1 to 4 and having peptidase activity are also included in the proteins having high dipeptide-degrading activity.

The homology among amino acid sequences and nucleotide sequences can be determined by using algorithm BLAST by Karlin and Altschul [Proc. Natl. Acad. Sci. USA, 90, 5873 (1993)] and FASTA [Methods Enzymol., 183, 63 (1990)]. On the basis of the algorithm BLAST, programs such as BLASTN and BLASTX have been developed [J. Mol. Biol., 215, 403 (1990)]. When a nucleotide sequence is analyzed by BLASTN on the basis of BLAST, the parameters, for instance, are as follows: score=100 and wordlength=12. When an amino acid sequence is analyzed by BLASTX on the basis of BLAST, the parameters, for instance, are as follows: score=50 and wordlength=3. When BLAST and Gapped BLAST programs are used, default parameters of each program are used. The specific techniques for these analyses are known.

The expression “the activity of a peptide-transporting protein is reduced” means that the peptide-transporting activity is reduced compared with a peptide-transporting protein having none of the above deletions, substitutions and additions of nucleotides.

The peptide-transporting activity of a microorganism can be measured by allowing a peptide as a substrate and microorganism cells to be present in an aqueous medium, thereby performing peptide-transporting reaction, and then determining the amount of the remaining peptide by a known method, e.g., HPLC analysis.

The above peptide-transporting proteins may be any proteins involved in peptide transporation of microorganisms, for example, proteins encoded by genes forming an operon on chromosomal DNA which form a complex on cell membrane to express dipeptide-transporting activity and those which have peptide-transporting activity as individual proteins. Preferred are proteins having high peptide-transporting activity.

Examples of the peptide-transporting proteins include: those existing in Escherichia coli such as DppA having the amino acid sequence shown in SEQ ID NO: 5, DppB having the amino acid sequence shown in SEQ ID NO: 6, DppC having the amino acid sequence shown in SEQ ID NO: 7, DppD having the amino acid sequence shown in SEQ ID NO: 8, DppF having the amino acid sequence shown in SEQ ID NO: 9, OppA (GenBank AAC76569), OppB (GenBank AAC76568), OppC (GenBank AAC76567), OppD (GenBank AAC76566), OppF (GenBank AAC76565), YddO (GenBank AAC74556), YddP (GenBank AAC74557), YddQ (GenBank AAC74558), YddR (GenBank AAC74559), YddS (GenBank AAC74560), YbiK (GenBank AAC73915), MppA (GenBank AAC74411), SapA (GenBank AAC74376), SapB (GenBank AAC74375), SapC (GenBank AAC74374), SapD (GenBank AAC74373) and SapF (GenBank AAC74372); those existing in Bacillus subtilis such as DppA (GenBank CAA40002), DppB (GenBank CAA40003), DppC (GenBank CAA40004), DppD (GenBank CAA40005), DppE (GenBank CAA40006), OppA (GenBank CAA39787), OppB (GenBank CAA39788), OppC (GenBank CAA39789), OppD (GenBank CAA39790), OppF (GenBank CAA39791), AppA (GenBank CAA62358), AppB (GenBank CAA62359), AppC (GenBank CAA62360), AppD (GenBank CAA62356), AppF (GenBank CAA62357), YclF (GenBank CAB12175) and YkfD (GenBank CAB13157); those existing in Corynebacterium glutamicum such as proteins having the amino acid sequences represented by BAB99048, BAB99383, BAB99384, BAB99385, BAB99713, BAB99714, BAB99715, BAB99830, BAB99831 and BAB99832 (registration Nos. of DNA Data Bank of Japan); and those existing in Saccharomyces cerevisiae such as OPT1 (GenBank NP_{—}012323), OPT2 (GenBank NP_{—}015520) and PTR2 (GenBank CAA82172). Examples of the proteins having high peptide-transporting activity include DppA, DppB, DppC, DppD and DppF having the amino acid sequences shown in SEQ ID NOS: 5 to 9, and proteins having amino acid sequences which have 80% or more, preferably 90% or more, more preferably 95% or more homology to the amino acid sequence shown in any of SEQ ID NOS: 5 to 9.

The homology among amino acid sequences can be determined by using programs such as BLAST and FASTA described above.

The microorganisms in which the activities of three or more kinds of peptidases are reduced or lost include microorganisms in which the activities of three or more arbitrary kinds of peptidases are reduced or lost provided that the microorganisms can normally grow, specifically, microorganisms in which the activities of preferably three to nine kinds, more preferably three to six kinds, further preferably three or four kinds of peptidases are reduced or lost.

Examples of peptidases include the above-described peptidases and dipeptidases existing in Escherichia coli, Bacillus subtilis, Corynebacterium glutamicum and Saccharomyces cerevisiae. Proteins consisting of amino acid sequences which have 80% or more, preferably 90% or more, more preferably 95% or more homology to the amino acid sequence shown in any of SEQ ID NOS: 1 to 4 and having peptidase activity are also included in the proteins having high dipeptide-degrading activity.

The homology among amino acid sequences can be determined by using programs such as BLAST and FASTA described above.

There is not any specific restriction as to the microorganism having the ability to produce a dipeptide so long as it has the ability to produce a dipeptide. Suitable microorganisms include microorganisms producing proteins having the activity to synthesize a dipeptide by condensation and ligation of one or more kinds of amino acids, microorganisms producing proteins having the activity to synthesize a dipeptide from an L-amino acid ester and an L-amino acid, and microorganisms producing proteins having the activity to synthesize a dipeptide from an L-amino acid amide and an L-amino acid.

The microorganisms producing proteins having the activity to synthesize a dipeptide by condensation and ligation of one or more kinds of amino acids include microorganisms producing a protein selected from the group consisting of NRPS, D-Ala-D-Ala ligase and bacilysin synthetase.

Examples of the microorganisms producing NRPS include procaryotes such as microorganisms of the genus Bacillus, eucaryotes such as microorganisms of the genus Penicillium, microorganisms producing BacA, BacB and BacC (GenBank AF007865), microorganisms producing TycA, TycB and TycC (GenBank AF004835), microorganisms producing PcbAB (GenBank M57425), and microorganisms producing a protein having an amino acid sequence which has 80% or more, preferably 90% or more, more preferably 95% or more homology to the amino acid sequence of any protein selected from BacA, BacB, BacC, TycA, TycB, TycC and PcbAB and having NRPS activity.

Examples of the microorganisms producing D-Ala-D-Ala ligase include procaryotes forming peptidoglycans, microorganisms producing DdlA (GenBank accession No. M58467), microorganisms producing DdlB (GenBank accession No. AE000118), microorganisms producing DdlC (GenBank accession No. D88151), and microorganisms producing a protein consisting of an amino acid sequence wherein one or more amino acid residues are deleted, substituted or added in the amino acid sequence of any protein selected from DdlA, DdlB and DdlC and having D-Ala-D-Ala ligase activity.

The homology among amino acid sequences can be determined by using programs such as BLAST and FASTA described above.

Examples of the microorganisms producing bacilysin synthetase include microorganisms belonging to the genus Bacillus, preferably, Bacillus subtilis, Bacillus amyloliquefaciens, Bacillus coagulans, Bacillus licheniformis, Bacillus megaterium and Bacillus pumilus, and microorganisms producing a protein selected from the following [1] to [4]:

[1] a protein having the amino acid sequence shown in any of SEQ ID NOS: 19 to 25 and 68;
[2] a protein consisting of an amino acid sequence wherein one or more amino acid residues are deleted, substituted or added in the amino acid sequence shown in any of SEQ ID NOS: 19 to 25 and 68 and having the activity to synthesize a dipeptide;
[3] a protein consisting of an amino acid sequence which has 65% or more homology to the amino acid sequence shown in any of SEQ ID NOS: 19 to 25 and 68 and having the activity to synthesize a dipeptide; and
[4] a protein having an amino acid sequence which has 80% or more homology to the amino acid sequence shown in SEQ ID NO: 33 and having the activity to synthesize a dipeptide.

The microorganisms producing proteins having the activity to synthesize a dipeptide from an L-amino acid ester and an L-amino acid include microorganisms producing proline iminopeptidase, specifically, those belonging to the genera Bacillus, Corynebacterium and Pseudomonas. Examples of such microorganisms are Bacillus subtilis ATCC 6633, Bacillus coagulans EK01 [J. Bacteriol., 174, 7919 (1992)], Corynebacterium glutamicum ATCC 13286, Pseudomonas putida AJ-2402 (FERM BP-8101), Pseudomonas putida ATCC 12633 and Pseudomonas putida AJ-2048 (FERM BP-8123) (microorganisms described in WO03/010307). The microorganisms producing proline iminopeptidase also include Arthrobacter nicotianae [FEMS Microbiol. Lett., 78, 191 (1999)], Escherichia coli (Japanese Published Unexamined Patent Application No. 113887/90), Flavobacterium meningosepticum [Arch. Biochem. Biophys., 336, 35 (1996)], Hafnia alvei [J. Biochem., 119, 468 (1996)], Lactobacillus delbrueckii [Microbiology, 140, 527 (1994)], Bacillus coagulans [J. Bacteriol., 174, 7919 (1994)], Aeromonas sobria [J. Biochem., 116, 818 (1994)], Xanthomonas campestris (Japanese Published Unexamined Patent Application No. 121860/97), Neisseria gonorrhoeae [Mol. Microbiol., 9, 1203 (1993)], Propionibacterium freudenreichii [Appl. Environ. Microbiol., 64, 4736 (1998)], Serratia marcescens [J. Biochem., 122, 601 (1997)], Corynebacterium variabilis [J. Appl. Microbiol., 90, 449 (2001)], Thermoplasma acidophilum [FEBS Lett., 398, 101 (1996)] and Pseudomonas aeruginosa [Nature, 406, 959 (2000)].

Further, the microorganisms producing proline iminopeptidase include microorganisms having the ability to produce a protein selected from the following [1] to [3]:

[1] proline iminopeptidase described in any of WO03/010307; FEMS Microbiol. Lett., 78, 191 (1999); Japanese Published Unexamined Patent Application No. 113887/90; Arch. Biochem. Biophys., 336, 35 (1996); J. Biochem., 119, 468 (1996); Microbiology, 140, 527 (1994); J. Bacteriol., 174, 7919 (1994); J. Biochem., 116, 818 (1994); Japanese Published Unexamined Patent Application No. 121860/97; Mol. Microbiol., 9, 1203 (1993); Appl. Environ. Microbiol., 64, 4736 (1998); J. Biochem., 122, 601 (1997); FEBS Lett., 398, 101 (1996); and Nature, 406, 959 (2000);
[2] a protein consisting of an amino acid sequence wherein one or more amino acid residues are deleted, substituted or added in the amino acid sequence of any proline iminopeptidase of the above [1] and having proline iminopeptidase activity; and
[3] a protein consisting of an amino acid sequence which has 80% or more homology to the amino acid sequence of any proline iminopeptidase of the above [1] and having proline iminopeptidase activity.

The microorganisms producing proteins having the activity to synthesize a dipeptide from an L-amino acid amide and an L-amino acid include microorganisms producing L-amino acid amide hydrolase, specifically, those belonging to the genera Bacillus, Corynebacterium, Erwinia, Rhodococcus, Chryseobacterium, Micrococcus, Pseudomonas, Cryptococcus, Trichosporon, Rhodosporidium, Sporobolomyces, Tremella, Torulaspora, Sterigmatomyces and Rhodotolura. Preferred are microorganisms belonging to the genera Bacillus, Corynebacterium and Pseudomonas. More preferred examples are Bacillus megaterium AJ3284 (FERM BP-8090), Corynebacterium glutamicum ATCC 13286, Micrococcus luteus ATCC 9341 and Pseudomonas saccharophila ATCC 15946 (microorganisms described in WO03/010187).

The microorganisms producing proteins having L-amino acid amide hydrolase activity include microorganisms having the ability to produce a protein of the following [1] or [2]:

[1] a protein consisting of an amino acid sequence wherein one or more amino acid residues are deleted, substituted or added in the amino acid sequence of L-amino acid amide hydrolase described in WO03/010187 and having L-amino acid amide hydrolase activity;
[2] a protein consisting of an amino acid sequence which has 80% or more homology to the amino acid sequence of L-amino acid amide hydrolase described in WO03/010187 and having L-amino acid amide hydrolase activity.

The above protein consisting of an amino acid sequence wherein one or more amino acid residues are deleted, substituted or added and having the activity to synthesize a dipeptide can be obtained, for example, by introducing a site-directed mutation into DNA encoding a protein selected from a protein consisting of the amino acid sequence shown in any of SEQ ID NOS: 19 to 25 and 68, a protein having proline iminopeptidase activity and a protein having L-amino acid amide hydrolase activity by site-directed mutagenesis described in Molecular Cloning, A Laboratory Manual, Second Edition, Cold Spring Harbor Laboratory Press (1989) (hereinafter referred to as Molecular Cloning, Second Edition); Current Protocols in Molecular Biology, John Wiley & Sons (1987-1997) (hereinafter referred to as Current Protocols in Molecular Biology); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc.

The number of amino acid residues which are deleted, substituted or added is not specifically limited, but is within the range where deletion, substitution or addition is possible by known methods such as the above site-directed mutagenesis. The suitable number is 1 to dozens, preferably 1 to 20, more preferably 1 to 10, further preferably 1 to 5.

The expression “one or more amino acid residues are deleted, substituted or added in any of the amino acid sequences shown in SEQ ID NOS: 19 to 25 and 68 and the amino acid sequences of a protein having proline iminopeptidase activity and a protein having L-amino acid amide hydrolase activity” means that the amino acid sequence may contain deletion, substitution or addition of a single or plural amino acid residues at an arbitrary position therein.

Deletion, substitution and addition may be simultaneously contained in one sequence, and amino acids to be substituted or added may be either natural or not. Examples of the natural amino acids are L-alanine, L-asparagine, L-aspartic acid, L-arginine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine.

The following are examples of the amino acids capable of mutual substitution. The amino acids in the same group can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

There is not any specific restriction as to the position where the above deletion, substitution or addition of one or more amino acid residues is introduced, so long as a protein having an amino acid sequence carrying the introduced mutation has the dipeptide-synthesizing activity. Suitable examples include amino acid residues which are not conserved in one or more amino acid sequences among the amino acid sequences shown in SEQ ID NOS: 19 to 25 and 68 when these sequences are compared.

The above proteins consisting of an amino acid sequence wherein one or more amino acid residues are deleted, substituted or added and having the activity to synthesize a dipeptide include a protein consisting of an amino acid sequence which has usually 65% or more, preferably 80% or more, more preferably 90% or more, particularly preferably 95% or more homology to the amino acid sequence shown in any of SEQ ID NOS: 19 to 25 and 68, and a protein consisting of an amino acid sequence which has usually 80% or more, preferably 90% or more, more preferably 95% or more homology to the amino acid sequence of proline iminopeptidase or L-amino acid amide hydrolase.

The homology among amino acid sequences and nucleotide sequences can be determined by using programs such as BLAST and FASTA described above.

The amino acid sequence shown in SEQ ID NO: 33 is a region conserved among the proteins having the amino acid sequences shown in SEQ ID NOS: 19 to 25 and is also a region corresponding to the consensus sequence of proteins having Ala-Ala ligase activity derived from various microorganisms.

Microorganisms producing a protein having an amino acid sequence which has 80% or more, preferably 90% or more, further preferably 95% or more homology to the amino acid sequence shown in SEQ ID NO: 33 and having the activity to synthesize a dipeptide are also included in the dipeptide-producing microorganisms.

In order that the protein having an amino acid sequence which has 80% or more, preferably 90% or more, further preferably 95% or more homology to the amino acid sequence shown in SEQ ID NO: 33 may have the activity to synthesize a dipeptide, it is desirable that the homology of its amino acid sequence to the amino acid sequence shown in any of SEQ ID NOS: 19 to 25 is at least 80% or more, usually 90% or more, and particularly 95% or more.

The homology among amino acid sequences can be determined by using programs such as BLAST and FASTA described above.

The microorganisms of the present invention also include microorganisms carrying a recombinant DNA obtained by ligating, to a vector DNA, DNA encoding a protein having the activity to synthesize a dipeptide by condensation and ligation of one or more kinds of amino acids, DNA encoding a protein having the activity to synthesize a dipeptide from an L-amino acid ester and an L-amino acid, or DNA encoding a protein having the activity to synthesize a dipeptide from an L-amino acid amide and an L-amino acid.

Examples of the microorganisms include those belonging to the genera Escherichia, Bacillus, Corynebacterium and Saccharomyces.

The DNAs encoding a protein having the activity to synthesize a dipeptide by condensation and ligation of one or more kinds of amino acids include DNAs encoding NRPS, D-Ala-D-Ala ligase or bacilysin synthetase.

Examples of the DNAs encoding NRPS include DNAs encoding a protein selected from the group consisting of BacA, BacB, BacC, TycA, TycB, TycC and PcbAB.

Examples of the DNAs encoding D-Ala-D-Ala ligase include DNAs encoding a protein selected from the group consisting of DdlA, DdlB and DdlC.

Examples of the DNAs encoding bacilysin synthetase include DNAs encoding proteins of the following [1] to [4]:

[1] a protein having the amino acid sequence shown in any of SEQ ID NOS: 19 to 25 and 68;
[2] a protein consisting of an amino acid sequence wherein one or more amino acid residues are deleted, substituted or added in the amino acid sequence shown in any of SEQ ID NOS: 19 to 25 and 68 and having the activity to synthesize a dipeptide;
[3] a protein consisting of an amino acid sequence which has 65% or more homology to the amino acid sequence shown in any of SEQ ID NOS: 19 to 25 and 68 and having the activity to synthesize a dipeptide; and
[4] a protein having an amino acid sequence which has 80% or more homology to the amino acid sequence shown in SEQ ID NO: 33 and having the activity to synthesize a dipeptide; and DNAs of the following [5] to [7]:
[5] DNA having the nucleotide sequence shown in any of SEQ ID NOS: 26 to 32, 64 and 65;
[6] DNA which hybridizes with DNA having the nucleotide sequence shown in any of SEQ ID NOS: 26 to 32, 64 and 65 under stringent conditions and which encodes a protein having the activity to synthesize a dipeptide; and
[7] DNA having a nucleotide sequence which has 80% or more homology to the nucleotide sequence shown in SEQ ID NO: 34 and encoding a protein having the activity to synthesize a dipeptide.

Examples of the DNAs encoding a protein having the activity to synthesize a dipeptide from an L-amino acid ester and an L-amino acid include DNAs encoding proteins of the following [1] to [3]:

[1] proline iminopeptidase described in any of WO03/010307; FEMS Microbiol. Lett., 78, 191 (1999); Japanese Published Unexamined Patent Application No. 113887/90; Arch. Biochem. Biophys., 336, 35 (1996); J. Biochem., 119, 468 (1996); Microbiology, 140, 527 (1994); J. Bacteriol., 174, 7919 (1994); J. Biochem., 116, 818 (1994); Japanese Published Unexamined Patent Application No. 121860/97; Mol. Microbiol., 9, 1203 (1993); Appl. Environ. Microbiol., 64, 4736 (1998); J. Biochem., 122, 601 (1997); FEBS Lett., 398, 101 (1996); and Nature, 406, 959 (2000);
[2] a protein consisting of an amino acid sequence wherein one or more amino acid residues are deleted, substituted or added in the amino acid sequence of any proline iminopeptidase of the above [1] and having proline iminopeptidase activity; and
[3] a protein consisting of an amino acid sequence which has 80% or more homology to the amino acid sequence of any proline iminopeptidase of the above [1] and having proline iminopeptidase activity; and DNAs of the following [4] and [5]:
[4] DNA encoding proline iminopeptidase and having the nucleotide sequence described in any of WO03/010307; FEMS Microbiol. Lett., 78, 191 (1999); Japanese Published Unexamined Patent Application No. 113887/90; Arch. Biochem. Biophys., 336, 35 (1996); J. Biochem., 119, 468 (1996); Microbiology, 140, 527 (1994); J. Bacteriol., 174, 7919 (1994); J. Biochem., 116, 818 (1994); Japanese Published Unexamined Patent Application No. 121860/97; Mol. Microbiol., 9, 1203 (1993); Appl. Environ. Microbiol., 64, 4736 (1998); J. Biochem., 122, 601 (1997); FEBS Lett., 398, 101 (1996); and Nature, 406, 959 (2000); and
[5] DNA which hybridizes with any DNA encoding proline iminopeptidase of the above [4] under stringent conditions and which encodes a protein having proline iminopeptidase activity.

Examples of the DNAs encoding a protein having the activity to synthesize a dipeptide from an L-amino acid amide and an L-amino acid include DNAs encoding proteins of the following [1] and [2]:

[1] a protein consisting of an amino acid sequence wherein one or more amino acid residues are deleted, substituted or added in the amino acid sequence of L-amino acid amide hydrolase described in WO03/010187 and having L-amino acid amide hydrolase activity; and
[2] a protein consisting of an amino acid sequence which has 80% or more homology to the amino acid sequence of L-amino acid amide hydrolase described in WO03/010187 and having L-amino acid amide hydrolase activity;
and DNAs of the following [3] and [4]:
[3] DNA having the nucleotide sequence described in WO03/010187 and encoding L-amino acid amide hydrolase; and
[4] DNA which hybridizes with DNA consisting of the nucleotide sequence described in WO03/010187 and encoding L-amino acid amide hydrolase under stringent conditions and which encodes a protein having L-amino acid amide hydrolase activity.

The above DNA capable of hybridization under stringent conditions refers to DNA which is obtained by colony hybridization, plaque hybridization, Southern blot hybridization, or the like using a part or the whole of any of the above DNAs as a probe. A specific example of such DNA is DNA which can be identified by performing hybridization at 65° C. in the presence of 0.7 to 1.0 mol/l, preferably 0.9 mol/l sodium chloride using a filter with colony- or plaque-derived DNA immobilized thereon, and then washing the filter at 65° C. with a 0.1 to 2-fold conc., preferably 0.1-fold conc. SSC solution (1-fold conc. SSC solution: 150 mmol/l sodium chloride and 15 mmol/l sodium citrate). Hybridization can be carried out according to the methods described in Molecular Cloning, Second Edition; Current Protocols in Molecular Biology; DNA Cloning 1: Core Techniques, A Practical Approach, Second Edition, Oxford University (1995), etc. Specifically, the hybridizable DNA includes DNA having at least 80% or more homology, preferably 90% or more homology, more preferably 95% or more homology to the nucleotide sequence of any of the above DNAs as calculated by use of a program such as BLAST or FASTA described above based on the above parameters.

It is possible to confirm that the DNA hybridizing with the above DNA under stringent conditions is DNA encoding a protein having the activity to synthesize a dipeptide in the following manner. That is, a recombinant DNA expressing the DNA is prepared and the recombinant DNA is introduced into a host cell to obtain a microorganism to be used as an enzyme source. Then, 1) the enzyme source and one or more kinds of amino acids are allowed to be present in an aqueous medium, followed by HPLC analysis or the like to know whether a dipeptide is formed and accumulated in the aqueous medium, 2) the enzyme source, an L-amino acid ester and an L-amino acid are allowed to be present in an aqueous medium, followed by HPLC analysis or the like to know whether a dipeptide is formed and accumulated in the aqueous medium, or 3) the enzyme source, an L-amino acid amide and an L-amino acid are allowed to be present in an aqueous medium, followed by HPLC analysis or the like to know whether a dipeptide is formed and accumulated in the aqueous medium.

The homology among nucleotide sequences can be determined by using programs such as BLAST and FASTA described above.

### 2. Methods for Preparing the Microorganisms of the Present Invention

The microorganisms of the present invention can be obtained by any of the following methods: 1) methods of imparting the dipeptide-producing ability to microorganisms in which the functions of one or more kinds of peptidases and one or more kinds of proteins having peptide-transporting activity are reduced or lost, or microorganisms in which the functions of three or more kinds of peptidases are reduced or lost; and 2) methods of reducing or causing loss of the functions of a) one or more kinds of peptidases and one or more kinds of peptide-transporting proteins or b) three or more kinds of peptidases of microorganisms having the ability to produce a dipeptide.

(1) Methods of Imparting the Dipeptide-Producing Ability to Microorganisms in which the Functions of One or More Kinds of Peptidases and One or More Kinds of Peptide-Transporting Proteins are Reduced or Lost, or Microorganisms in which the Functions of Three or More Kinds of Peptidases are Reduced or Lost
(i) Preparation of Microorganisms in which the Activities of Peptidases and Peptide-Transporting Proteins are Reduced or Lost

The microorganisms in which the activities of peptidases and peptide-transporting proteins are reduced or lost may be obtained by any method capable of preparing such microorganisms. For example, they can be obtained by introducing a deletion, substitution or addition of a nucleotide into peptidase genes and genes encoding peptide-transporting proteins on chromosomal DNAs of microorganisms as described below.

The methods for introducing a deletion, substitution or addition of a nucleotide into a gene on the chromosomal DNA of a microorganism include methods utilizing homologous recombination. An example of the methods utilizing general homologous recombination is a method using a plasmid for homologous recombination prepared by ligating a mutant gene having an introduced nucleotide deletion, substitution or addition with a plasmid DNA incapable of autonomous replication in a host cell into which the nucleotide deletion or the like is to be introduced and carrying a drug resistance gene.

The plasmid for homologous recombination is introduced into a host cell by an ordinary method, followed by selection of a transformant in which the plasmid for homologous recombination has been integrated into the chromosomal DNA by homologous recombination using the drug resistance as a marker. The obtained transformant is cultured using a medium which does not contain the drug for several hours to one day, and then spread on an agar medium containing the drug and on an agar medium without the drug. By selecting a strain which does not grow on the former medium but can grow on the latter medium, the strain in which second homologous recombination occurred on the chromosomal DNA can be obtained. Introduction of a nucleotide deletion, substitution or addition into a desired gene on the chromosomal DNA can be confirmed by determining the nucleotide sequence of a region of the chromosomal DNA containing the gene into which the deletion or the like has been introduced.

By use of the above method, a nucleotide deletion, substitution or addition can be introduced into desired genes on chromosomal DNAs of microorganisms such as those belonging to the genera Escherichia, Bacillus, Corynebacterium and Saccharomyces.

Further, a nucleotide deletion, substitution or addition can be efficiently introduced into plural genes by utilizing homologous recombination according to a method using a linear DNA.

Specifically, a linear DNA containing a gene into which a nucleotide deletion, substitution or addition is to be introduced is incorporated into a cell to cause homologous recombination between chromosomal DNA and the introduced linear DNA. This method is applicable to any microorganisms capable of efficiently incorporating a linear DNA. Preferred microorganisms are those belonging to the genera Escherichia and Bacillus. Escherichia coli is more preferred, and Escherichia coli expressing a group of recombinant proteins derived from λ phage (Red recombination system) is further preferred.

An example of Escherichia coli expressing λ Red recombination system is Escherichia coli JM101 carrying pKD46, which is a plasmid DNA comprising a λ Red recombination system gene (available from Escherichia coli Genetic Stock Center, Yale University, U.S.A.)

Examples of the DNAs useful for homologous recombination are as follows:

(a) linear DNA in which DNAs present on the outside of a region of chromosomal DNA to be subjected to introduction of a nucleotide deletion, substitution or addition or DNAs having homology to the DNAs are present at both termini of a drug resistance gene;
(b) linear DNA in which DNAs present on the outside of a region of chromosomal DNA to be subjected to introduction of a nucleotide deletion, substitution or addition or DNAs having homology to the DNAs are directly ligated with each other;
(c) linear DNA in which DNAs present on the outside of a region of chromosomal DNA to be subjected to introduction of a nucleotide deletion, substitution or addition or DNAs having homology to the DNAs are present at both termini of a drug resistance gene and a gene that can be used for negative selection; and
(d) linear DNA of the above (a) in which a nucleotide sequence recognized by yeast-derived Flp recombinase [Proc. Natl. Acad. Sci. USA., 82, 5875 (1985)] is additionally present between the drug resistance gene and DNAs present on the outside of a region of chromosomal DNA or DNAs having homology to the DNAs.

As the drug resistance gene, any drug resistance genes that impart resistance to a drug to which the host microorganism shows sensitivity can be used. When Escherichia coli is used as the host microorganism, examples of the drug resistance genes are kanamycin resistance gene, chloramphenicol resistance gene, gentamicin resistance gene, spectinomycin resistance gene, tetracycline resistance gene and ampicillin resistance gene.

The “gene that can be used for negative selection” refers to a gene that is fatal to a host microorganism under certain culture conditions when the gene is expressed in the host microorganism. Examples of the genes are sacB gene derived from a microorganism belonging to the genus Bacillus [Appl. Environ. Microbiol., 59, 1361-1366 (1993)] and rpsL gene derived from a microorganism belonging to the genus Escherichia [Genomics, 72, 99-104 (2001)].

The DNAs present on the outside of a region of chromosomal DNA to be subjected to introduction of a substitution or deletion or DNAs having homology to the DNAs in the above linear DNAs are located in the same direction as that on the chromosomal DNA, and their length is preferably about 10 by to 100 bp, more preferably about 20 by to 50 bp, and further preferably about 30 by to 40 bp.

The nucleotide sequence recognized by yeast-derived Flp recombinase is not specifically limited so long as it is a nucleotide sequence recognized by the said protein and catalyzing homologous recombination. Preferred examples are DNA having the nucleotide sequence shown in SEQ ID NO: 39, and DNA having a nucleotide sequence wherein one to several nucleotides are deleted, substituted or added in the said DNA and having a nucleotide sequence recognized by yeast-derived Flp recombinase and catalyzing homologous recombination.

The “DNA having homology” refers to DNA having such a degree of identity that allows occurrence of homologous recombination between the subject region of chromosomal DNA and the above linear DNA, specifically, DNA having 80% or more homology, preferably 90% or more homology, more preferably 95% or more homology, further preferably 100% homology.

The homology among nucleotide sequences can be determined by using programs such as BLAST and FASTA described above.

The above linear DNA fragments can be prepared by PCR. The desired linear DNA can also be obtained by constructing DNA containing the above linear DNA on plasmid and then carrying out treatment with restriction enzymes.

Examples of the methods for introducing a nucleotide deletion, substitution or addition into the chromosomal DNA of a microorganism include the following Methods 1 to 4.

### Method 1:

A method which comprises introducing the linear DNA of the above (a) or (d) into a host microorganism and selecting a transformant carrying the linear DNA inserted on its chromosomal DNA by homologous recombination using the drug resistance as a marker.

### Method 2:

A method which comprises introducing the DNA, in which DNAs present on the outside of a region of chromosomal DNA to be subjected to introduction of a nucleotide deletion, substitution or addition or DNAs having homology to the DNAs are directly ligated with each other, into the transformant obtained according to the above Method 1 and eliminating the drug resistance gene inserted on its chromosomal DNA according to the method to substitute or delete a region of the chromosomal DNA of the microorganism.

### Method 3:

A method which comprises:

[1] introducing the linear DNA of the above (c) into a host microorganism and selecting a transformant carrying the linear DNA inserted on its chromosomal DNA by homologous recombination using the drug resistance as a marker;
[2] synthesizing DNA by ligating DNAs having homology to the DNAs present on the outside of a region of chromosomal DNA to be subjected to introduction of a substitution or deletion in the same direction as that on the chromosomal DNA, and introducing the synthesized DNA into the transformant obtained in the above [1]; and
[3] culturing the transformant subjected to the operation of the above [2] under conditions such that the gene that can be used for negative selection is expressed, and selecting a strain capable of growing by the culturing as a strain in which the drug resistance gene and the gene that can be used for negative selection are eliminated from the chromosomal DNA.

### Method 4:

A method which comprises:

[1] introducing the linear DNA of the above (d) into a host microorganism and selecting a transformant carrying the linear DNA inserted on its chromosomal DNA by homologous recombination using the drug resistance as a marker; and
[2] introducing a Flp recombinase gene expression plasmid into the transformant obtained in the above [1], and after expression of the gene, obtaining a strain sensitive to the drug used in the above [1].

In the above methods, introduction of the linear DNA into a host microorganism can be carried out by any of the methods for introducing DNA into the microorganism, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

By using a DNA in which an arbitrary gene to be inserted to chromosomal DNA is incorporated in the center part of the DNA used in Method 2 or Method 3 [2], it is possible to eliminate the drug resistance gene and at the same time to insert an arbitrary gene to the chromosomal DNA.

The above Methods 2 to 4 are methods that leave no foreign genes such as a drug resistance gene and a gene usable for negative selection on the chromosomal DNA of the transformant to be finally obtained. Therefore, it is possible to readily produce a microorganism having nucleotide deletions, substitutions or additions in two or more different regions of the chromosomal DNA by repeating the operations of Method 2, Method 3 [1] to [3], and Method 4 [1] and [2] using the same drug resistance gene and the same gene usable for negative selection.

### (ii) Methods of Imparting the Dipeptide-Producing Ability

The methods of imparting the dipeptide-producing ability to the microorganisms used in the above (i) include the following methods.

### (a) Preparation of DNA Encoding a Protein Having the Activity to Synthesize a Dipeptide

The above DNA encoding a protein having the activity to synthesize a dipeptide can be obtained by the methods described below utilizing nucleotide sequence information on the DNA.

For example, the DNA encoding bacilysin synthetase can be obtained by Southern hybridization of a chromosomal DNA library from a microorganism belonging to the genus Bacillus using a probe designed based on the nucleotide sequence shown in any of SEQ ID NOS: 26 to 32, 64 and 65, or by PCR [PCR Protocols, Academic Press (1990)] using primer DNAs designed based on the nucleotide sequence shown in any of SEQ ID NOS: 26 to 32, 64 and 65 and, as a template, the chromosomal DNA of a microorganism belonging to the genus Bacillus.

The DNA encoding a protein having the activity to synthesize a dipeptide can also be obtained by conducting a search through various gene sequence databases for a sequence having 65% or more homology, preferably 80% or more homology, more preferably 90% or more homology, further preferably 95% or more homology to the nucleotide sequence of DNA encoding the amino acid sequence shown in any of SEQ ID NOS: 19 to 25, 33 and 68, and obtaining the desired DNA, based on the nucleotide sequence obtained by the search, from a chromosomal DNA or cDNA library of an organism having the nucleotide sequence according to the above-described method.

The obtained DNA, as such or after cleavage with appropriate restriction enzymes, is inserted into a vector by a conventional method to obtain a recombinant DNA. A plasmid DNA is extracted from a transformant obtained by introducing the recombinant DNA into Escherichia coli. Then, the nucleotide sequence of the DNA can be determined by a conventional sequencing method such as the dideoxy method [Proc. Natl. Acad. Sci., USA, 74, 5463 (1977)] or by using a nucleotide sequencer such as 373A DNA Sequencer (Perkin-Elmer Corp.).

In cases where the obtained DNA is found to be a partial DNA by the analysis of nucleotide sequence, the full length DNA can be obtained by Southern hybridization of a chromosomal DNA library using the partial DNA as a probe.

It is also possible to prepare the desired DNA by chemical synthesis using a DNA synthesizer (e.g., Model 8905, PerSeptive Biosystems) based on the determined nucleotide sequence of the DNA.

Examples of the DNAs that can be obtained by the above-described method are DNAs having the nucleotide sequences shown in SEQ ID NOS: 26 to 32, 64 and 65.

Examples of the vectors for inserting the DNA include pBluescriptII KS(+) (Stratagene), pDIRECT [Nucleic Acids Res., 18, 6069 (1990)], pCR-Script Amp SK(+) (Stratagene), pT7 Blue (Novagen, Inc.), pCR II (Invitrogen Corp.) and pCR-TRAP (Genhunter Corp.).

Examples of Escherichia coli include Escherichia coli XL1-Blue, Escherichia coli XL2-Blue, Escherichia coli DH1, Escherichia coli MC1000, Escherichia coli KY3276, Escherichia coli W1485, Escherichia coli JM101, Escherichia coli JM109, Escherichia coli HB101, Escherichia coli No. 49, Escherichia coli W3110, Escherichia coli NY49, Escherichia coli MP347, Escherichia coli NM522 and Escherichia coli ME8415.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

An example of the microorganism carrying the DNA encoding a protein having the dipeptide-synthesizing activity obtained by the above method is Escherichia coli NM522/pPE43, which is a microorganism carrying a recombinant DNA comprising DNA having the sequence shown in SEQ ID NO: 19 described below.

### (b) Production of a Protein Having the Dipeptide-Synthesizing Activity

The protein having the dipeptide-synthesizing activity can be produced by expressing the DNA obtained by the methods described in the above (a) in host cells using the methods described in Molecular Cloning, Second Edition, Current Protocols in Molecular Biology, etc., for example, in the following manner.

On the basis of the DNA obtained by the methods described in the above (a), a DNA fragment of an appropriate length comprising a region encoding the protein having the dipeptide synthesizing-activity is prepared according to need. The productivity of the protein can be enhanced by replacing a nucleotide in the nucleotide sequence of the region encoding the protein so as to make a codon most suitable for the expression in a host cell.

The DNA fragment is inserted downstream of a promoter in an appropriate expression vector to prepare a recombinant DNA.

A transformant producing the protein having the dipeptide synthesizing-activity can be obtained by introducing the recombinant DNA into a host cell suited for the expression vector.

As the host cell, any microorganisms such as bacterial cells and yeast cells that are capable of expressing the desired gene can be used.

The expression vectors that can be employed are those capable of autonomous replication or integration into the chromosome in the above host cells and comprising a promoter at a position appropriate for the transcription of the DNA encoding the protein having the dipeptide synthesizing-activity.

When a procaryote such as a bacterium is used as the host cell, it is preferred that the recombinant DNA comprising the DNA encoding a protein having the dipeptide-synthesizing activity is a recombinant DNA which is capable of autonomous replication in the procaryote and which comprises a promoter, a ribosome binding sequence, the DNA encoding a protein having the dipeptide-synthesizing activity, and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelixl (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 (Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Shuzo Co., Ltd.), pUC118 (Takara Shuzo Co., Ltd.) and pPA1 (Japanese Published Unexamined Patent Application No. 233798/88).

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, SPO1 promoter, SPO2 promoter and penP promoter can be used. Artificially designed and modified promoters such as a promoter in which two Pₜᵣₚs are combined in tandem, tac promoter, lacT7 promoter and letI promoter, etc. can also be used.

Also useful are promoters such as xylA promoter for the expression in bacteria belonging to the genus Bacillus [Appl. Microbiol. Biotechnol., 35, 594-599 (1991)] and P54-6 promoter for the expression in bacteria belonging to the genus Corynebacterium [Appl. Microbiol. Biotechnol., 53, 674-679 (2000)].

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA encoding the protein having the dipeptide synthesizing-activity is ligated to an expression vector, the transcription termination sequence is not essential, but it is preferred to place the transcription termination sequence immediately downstream of the structural gene.

An example of such recombinant DNA is pPE43 described below.

Examples of procaryotes suitable for use as host cells include microorganisms belonging to the genera Escherichia, Bacillus and Corynebacterium. Specific examples are Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM101, Escherichia coli JM109, Escherichia coli HB101, Escherichia coli No. 49, Escherichia coli W3110, Escherichia coli NY49, Escherichia coli MP347, Escherichia coli NM522, Bacillus subtilis ATCC 33712, Bacillus megaterium, Bacillus sp. FERM BP-6030, Bacillus amyloliquefaciens, Bacillus coagulans, Bacillus licheniformis, Bacillus pumilus, Corynebacterium glutamicum ATCC 13032 and Corynebacterium glutamicum ATCC 14297.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When a strain belonging to the genus Saccharomyces is used as the host cell, YEp13 (ATCC 37115), YEp24 (ATCC 37051), YCp50 (ATCC 37419), pHS19, pHS15, etc. can be used as the expression vector.

As the promoter, any promoters capable of functioning in strains belonging to the genus Saccharomyces can be used. Suitable promoters include PHO5 promoter, PGK promoter, GAP promoter, ADH promoter, gal 1 promoter, gal 10 promoter, heat shock polypeptide promoter, MFα1 promoter and CUP 1 promoter.

Examples of suitable host cells are strains belonging to the genus Saccharomyces, specifically, Saccharomyces cerevisiae.

Introduction of the recombinant DNA can be carried out by any of the methods for introducing DNA into yeast, for example, electroporation [Methods Enzymol., 194, 182 (1990)], the spheroplast method [Proc. Natl. Acad. Sci. USA, 81, 4889 (1984)] and the lithium acetate method [J. Bacteriol., 153, 163 (1983)].

### (2) Methods of Reducing or Causing Loss of the Functions of a) One or More Kinds of Peptidases and One or More Kinds of Peptide-Transporting Proteins or b) Three or More Kinds of Peptidases of Microorganisms Having the Ability to Produce a Dipeptide

Microorganisms having the ability to produce a dipeptide can be prepared by carrying out the methods described in the above (1) (ii) using arbitrary microorganisms as host cells. By carrying out the methods described in the above (1) (i) using the microorganisms thus prepared, microorganisms in which the functions of one or more kinds of peptidases and one or more kinds of peptide-transporting proteins or the functions of three or more kinds of peptidases are reduced or lost and which have the ability to produce a dipeptide can be prepared.

The microorganisms of the present invention can also be obtained by carrying out the methods described in the above (1) (i) using microorganisms inherently having the ability to produce a dipeptide.

Examples of the above microorganisms include those belonging to the genera Escherichia, Bacillus, Corynebacterium and Saccharomyces. Preferred are Escherichia coli, Bacillus subtilis, Corynebacterium glutamicum and Saccharomyces cerevisiae.

### 3. Process for Producing a Dipeptide of the Present Invention

The production processes of the present invention include:

(i) a process for producing a dipeptide, which comprises: allowing an enzyme source and one or more kinds of amino acids to be present in an aqueous medium, said enzyme source being a culture of the microorganism of the present invention or a treated matter of the culture; allowing the dipeptide to form and accumulate in the aqueous medium; and recovering the dipeptide from the medium;
(ii) a process for producing a dipeptide, which comprises: allowing an enzyme source, an L-amino acid ester and an L-amino acid to be present in an aqueous medium, said enzyme source being a culture of the microorganism of the present invention or a treated matter of the culture; allowing the dipeptide to form and accumulate in the aqueous medium; and recovering the dipeptide from the medium; and
(iii) a process for producing a dipeptide, which comprises: allowing an enzyme source, an L-amino acid amide and an L-amino acid to be present in an aqueous medium, said enzyme source being a culture of the microorganism of the present invention or a treated matter of the culture; allowing the dipeptide to form and accumulate in the aqueous medium; and recovering the dipeptide from the medium.

One or more kinds, preferably one or two kinds of amino acids used as substrates in the above production process (i) are amino acids, preferably amino acid selected from the group consisting of L-amino acids, glycine (Gly) and β-alanine (β-Ala), and can be used in any combination. Examples of L-amino acids are L-alanine (L-Ala), L-glutamine (L-Gln), L-glutamic acid (L-Glu), L-valine (L-Val), L-leucine (L-Leu), L-isoleucine (L-Ile), L-proline (L-Pro), L-phenylalanine (L-Phe), L-tryptophan (L-Trp), L-methionine (L-Met), L-serine (L-Ser), L-threonine (L-Thr), L-cysteine (L-Cys), L-asparagine (L-Asn), L-tyrosine (L-Tyr), L-lysine (L-Lys), L-arginine (L-Arg), L-histidine (L-His), L-aspartic acid (L-Asp), L-α-aminobutyric acid (L-α-AB), L-azaserine, L-theanine, L-4-hydroxyproline (L-4-HYP), L-3-hydroxyproline (L-3-HYP), L-ornithine (L-Orn), L-citrulline (L-Cit) and L-6-diazo-5-oxo-norleucine.

The amino acids which are more preferably used in the above process (i) include the following: a combination of one kind of amino acid selected from the group consisting of L-Ala, Gly, L-Met, L-Ser, L-Thr and β-Ala, and one kind of amino acid selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn, L-Cit and L-6-diazo-5-oxo-norleucine; a combination of L-Gln and L-Phe; and a combination of L-α-AB and L-Gln, L-Arg or L-α-AB. Further preferred amino acids are: a combination of L-Ala and one kind of amino acid selected from the group consisting of L-Ala, L-Gln, Gly, L-Val, L-Leu, L-Ile, L-Phe, L-Trp, L-Met, L-Ser, L-Thr, L-Cys, L-Asn, L-Tyr, L-Lys, L-Arg, L-His, L-α-AB, L-azaserine, L-Cit and L-theanine; a combination of Gly and one kind of amino acid selected from the group consisting of L-Gln, Gly, L-Phe, L-Trp, L-Met, L-Ser, L-Thr, L-Cys, L-Tyr, L-Lys, L-Arg, L-α-AB and L-Cit; a combination of L-Met and one kind of amino acid selected from the group consisting of L-Phe, L-Met, L-Ser, L-Thr, L-Cys, L-Tyr, L-Lys and L-His; a combination of L-Ser and one kind of amino acid selected from the group consisting of L-Gln, L-Phe, L-Ser, L-Thr, L-Tyr, L-His and L-α-AB; a combination of L-Thr and one kind of amino acid selected from the group consisting of L-Gln, L-Phe, L-Leu, L-Thr and L-α-AB; a combination of L-Gln and L-Phe; a combination of β-Ala and one kind of amino acid selected from the group consisting of L-Phe, L-Met, L-His and L-Cit; and a combination of L-α-AB and L-Gln, L-Arg or L-α-AB.

In the above production process (i), the L-amino acid used as a substrate is added to the aqueous medium at the start or in the course of reaction to give a concentration of 0.1 to 500 g/l, preferably 0.2 to 200 g/l.

The dipeptides produced by the above process (i) include the dipeptides represented by the following formula (I):

R¹-R² (I)

(wherein R¹ and R², which may be the same or different, each represent an amino acid). Preferred dipeptides are those represented by the above formula (I) wherein R¹ and R², which may be the same or different, each represent an amino acid selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-H is, L-Asp, L-α-AB, β-Ala, L-azaserine, L-theanine, L-4-HYP, L-3-HYP, L-Orn and L-6-diazo-5-oxo-norleucine. More preferred are dipeptides wherein R¹ is L-Ala, Gly, L-Met, L-Ser, L-Thr or β-Ala, and R² is L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn or L-6-diazo-5-oxo-norleucine. Further preferred dipeptides are: dipeptides wherein R¹ is L-Ala and R² is L-Gln, Gly, L-Val, L-Leu, L-Ile, L-Phe, L-Trp, L-Met, L-Ser, L-Thr, L-Cys, L-Asn, L-Tyr, L-Lys, L-Arg, L-His, L-α-AB, L-azaserine or L-theanine; dipeptides wherein R¹ is Gly and R² is L-Gln, Gly, L-Trp, L-Met, L-Ser, L-Thr, L-Cys, L-Tyr, L-Lys, L-Arg or L-α-AB; dipeptides wherein R¹ is L-Met and R² is L-Phe, L-Met, L-Cys, L-Tyr, L-Lys or L-His; dipeptides wherein R¹ is L-Ser and R² is L-Gln, Gly, L-Phe, L-Met, L-Ser, L-Thr, L-Tyr, L-His or L-α-AB; dipeptides wherein R¹ is L-Thr and R² is L-Gln, L-Gly, L-Phe, L-Met, L-Ser, L-Thr or L-α-AB; dipeptides wherein R¹ is L-Gln and R² is L-Phe or L-α-AB; a dipeptide wherein R¹ is L-Phe and R² is L-Gln; a dipeptide wherein R¹ is L-Trp and R² is Gly; dipeptides wherein R¹ is L-Cys and R² is L-Ala, L-Gln, Gly or L-Met; dipeptides wherein R¹ is L-Lys and R² is L-Ala, Gly or L-Met; a dipeptide wherein R¹ is L-Arg and R² is L-α-AB; a dipeptide wherein R¹ is L-His and R² is L-Met; and dipeptides wherein R¹ is L-α-AB and R² is L-Ala, L-Gln, Gly, L-Ser, L-Thr, L-Arg or L-α-AB.

Further, in the above process, compounds which can be metabolized by the microorganism of the present invention to produce ATP, for example, sugars such as glucose, alcohols such as ethanol, and organic acids such as acetic acid may be added, as ATP source, to the aqueous medium according to need.

The L-amino acid ester and L-amino acid used as substrates in the above production process (ii) may be any of L-amino acid esters and L-amino acids that can be used as substrates by the microorganism of the present invention to form a dipeptide, and they can be used in any combination. Preferably, the L-amino acid ester is selected from the group consisting of L-alanine ester, glycine ester, L-valine ester, L-isoleucine ester, L-methionine ester, L-phenylalanine ester, L-serine ester, L-threonine ester, L-glutamine ester, L-tyrosine ester, L-arginine ester, L-aspartic acid-α-ester, L-aspartic acid-β-ester, L-leucine ester, L-asparagine ester, L-lysine ester, L-aspartic acid-α,β-dimethyl ester and L-glutamine-γ-ester, and the L-amino acid is selected from the group consisting of L-Gln, L-Asn, Gly, L-Ala, L-Leu, L-Met, L-Pro, L-Phe, L-Trp, L-Ser, L-Thr, L-Tyr, L-Lys, L-Arg, L-His and L-Glu.

In the above process (ii), the L-amino acid ester and L-amino acid used as substrates are added to the aqueous medium at the start or in the course of reaction to give a concentration of 0.1 to 500 g/l, preferably 0.2 to 200 g/l.

The L-amino acid amide and L-amino acid used as substrates in the above production process (iii) may be any of L-amino acid amides and L-amino acids that can be used as substrates by the microorganism of the present invention to form a dipeptide, and they can be used in any combination. Preferably, the L-amino acid amide is selected from the group consisting of L-alanine amide, glycine amide and L-aspartic acid amide, and the L-amino acid is selected from the group consisting of L-Gln, L-Asn, Gly, L-Ala, L-Val, L-Leu, L-Ile, L-Met, L-Pro, L-Phe, L-Trp, L-Ser, L-Thr, L-Tyr, L-Lys, L-Arg, L-His and L-Glu.

In the above process (iii), the L-amino acid amide and L-amino acid used as substrates are added to the aqueous medium at the start or in the course of reaction to give a concentration of 0.1 to 500 g/l, preferably 0.2 to 200 g/l.

The aqueous medium used in the production processes of the present invention may comprise any components and may have any composition so far as the dipeptide-forming reaction is not inhibited. Suitable aqueous media include water and buffers such as phosphate buffer, carbonate buffer, acetate buffer, borate buffer, citrate buffer and Tris buffer. The aqueous medium may comprise alcohols such as methanol and ethanol, esters such as ethyl acetate, ketones such as acetone, and amides such as acetamide.

The dipeptide-forming reaction is carried out in the aqueous medium at pH 5 to 11, preferably pH 6 to 10, at 20 to 60° C., preferably 25 to 45° C., for 2 to 150 hours, preferably 6 to 120 hours.

If necessary, a surfactant or an organic solvent may further be added to the aqueous medium.

Any surfactant that promotes the formation of a dipeptide can be used. Suitable surfactants include nonionic surfactants such as polyoxyethylene octadecylamine (e.g., Nymeen S-215, NOF Corporation), cationic surfactants such as cetyltrimethylammonium bromide and alkyldimethylbenzylammonium chloride (e.g., Cation F2-40E, NOF Corporation), anionic surfactants such as lauroyl sarcosinate, and tertiary amines such as alkyldimethylamine (e.g., Tertiary Amine FB, NOF Corporation), which may be used alone or in combination. The surfactant is usually used at a concentration of 0.1 to 50 g/l. As the organic solvent, xylene, toluene, aliphatic alcohols, acetone, ethyl acetate, etc. may be used usually at a concentration of 0.1 to 50 ml/l.

The treated matters of the culture include concentrated culture, dried culture, cells obtained by centrifuging the culture, and products obtained by treating the cells by various means such as drying, freeze-drying, treatment with a surfactant, ultrasonication, mechanical friction, treatment with a solvent, enzymatic treatment and immobilization. The treated matters of the culture of the present invention also include crude extracts of protein obtained by removing insoluble matters and the like from the treated matters obtained by treating the above cells by means such as treatment with a surfactant, ultrasonication, mechanical friction, treatment with a solvent and enzymatic treatment.

When the culture or a treated matter of the culture is used as the enzyme source, the amount of the enzyme source to be added varies according to its specific activity, etc., but is, for example, 5 to 1000 mg, preferably 10 to 400 mg per mg of amino acid, L-amino acid ester or L-amino acid amide used as a substrate.

Recovery of the dipeptide formed and accumulated in the aqueous medium can be carried out by ordinary methods using active carbon, ion-exchange resins, etc. or by means such as extraction with an organic solvent, crystallization, thin layer chromatography and high performance liquid chromatography.

Further, the above production processes (ii) and (iii) can be carried out according to the descriptions in WO03/010189 or WO03/010187.

Experimental examples of the present invention are shown below.

### Experimental Example 1

### Acquisition of Proteins Having the Activity to Synthesize a Dipeptide and DNAs Encoding the Same

Construction of a Plasmid Expressing ywfE Gene Derived from Bacillus subtilis

A ywfE gene fragment of Bacillus subtilis was obtained in the following manner.

By using a DNA synthesizer (Model 8905, PerSeptive Biosystems, Inc.), DNAs having the nucleotide sequences shown in SEQ ID NOS: 35 and 36 (hereinafter referred to as primer A and primer B, respectively) were synthesized. Primer A has a nucleotide sequence containing a region wherein the initiation codon of ywfE gene (atg) is substituted by the NcoI recognition sequence (ccatgg). Primer B has a nucleotide sequence containing a region wherein the termination codon of ywfE gene is substituted by the BamHI recognition sequence (ggatcc).

PCR was carried out using the chromosomal DNA of Bacillus subtilis as a template and the above primer A and primer B as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

One-tenth of the resulting reaction mixture was subjected to agarose gel electrophoresis to confirm that a ca. 1.4 kb fragment corresponding to the ywfE gene fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained solution (5 μl) was subjected to reaction to cleave the amplified DNA with restriction enzymes NcoI and BamHI. DNA fragments were separated by agarose gel electrophoresis, and a 1.4 kb DNA fragment containing ywfE gene was recovered using GENECLEAN II Kit (Bio 101).

C-Terminal His-tagged recombinant expression vector pQE60 (Qiagen, Inc.) (0.2 μg) was cleaved with restriction enzymes NcoI and BamHI. DNA fragments were separated by agarose gel electrophoresis, and a 3.4 kb DNA fragment was recovered in the same manner as above.

The 1.4 kb DNA fragment containing ywfE gene and the 3.4 kb DNA fragment obtained above were subjected to ligation reaction using a ligation kit (Takara Shuzo Co., Ltd.) at 16° C. for 16 hours.

Escherichia coli NM522 (Stratagene) was transformed using the ligation reaction mixture according to the method using calcium ion [Proc. Natl. Acad. Sci. USA, 69, 2110 (1972)], spread on LB agar medium containing 50 μg/ml ampicillin, and cultured overnight at 30° C.

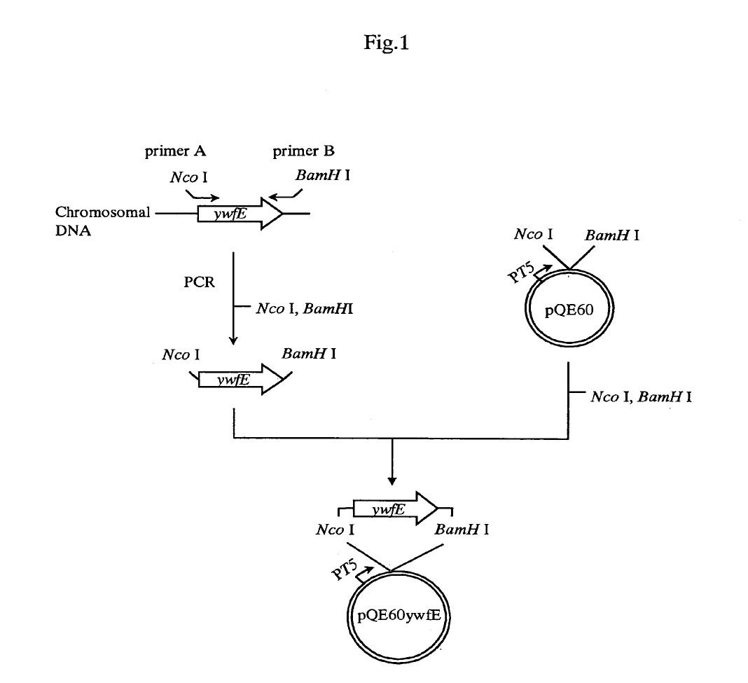

A plasmid was extracted from a colony of the transformant that grew on the medium according to a known method, whereby pQE60ywfE, which is a C-terminal His-tagged ywfE gene expression vector, was obtained. The structure of the vector was confirmed by digestion with restriction enzymes (FIG. 1).

### Experimental Example 2

### Acquisition of a ywfE Gene Product

Escherichia coli NM522/pQE60ywfE carrying pQE60ywfE was inoculated into 8 ml of LB medium containing 50 μg/ml ampicillin in a test tube, and cultured at 28° C. for 17 hours. The resulting culture was inoculated into 50 ml of LB medium containing 50 μg/ml ampicillin in a 250-ml Erlenmeyer flask, and cultured at 30° C. for 3 hours. Then, isopropyl-β-D-thiogalactopyranoside (IPTG) was added to give a final concentration of 1 mmol/l, followed by further culturing at 30° C. for 4 hours. The resulting culture was centrifuged to obtain wet cells, and a His-tagged recombinant enzyme was purified from the wet cells using HisTrap (His-tagged protein purification kit, Amersham Pharmacia Biotech) according to the instructions attached thereto.

### Experimental Example 3

### Production of Dipeptides Using the His-Tagged Recombinant Enzyme (1)

(i) A reaction mixture (0.1 ml) comprising 0.04 mg of the purified His-tagged recombinant enzyme obtained in Experimental Example 2, 100 mmol/l Tris-HCl (pH 8.0), 60 mmol/1 magnesium chloride, 60 mmol/l ATP, 30 mmol/l L-Ala and 30 mmol/l L-Gln was prepared, and reaction was carried out at 37° C. for 16 hours.

After the completion of reaction, the reaction product was derivatized by the dinitrophenol method and then analyzed by HPLC. The HPLC analysis was carried out using, as a separation column, Lichrosorb-RP-18 column (Kanto Kagaku) and, as an eluting solution, 1% (v/v) phosphoric acid and 25% (v/v) acetonitrile at a flow rate of 0.7 ml/min. As a result, it was confirmed that 3.7 g/l L-Ala-L-Gln and 0.3 g/l L-alanyl-L-alanine (L-Ala-L-Ala) were formed and accumulated in the reaction mixture.

(ii) Reactions were carried out under the same conditions as in the above (i) using reaction mixtures having the same composition as that of the reaction mixture of the above (i) except that 0.01 mg of the enzyme was used and L-Phe, L-Met, L-Leu and L-Val, respectively, were used in place of L-Gln.

After the completion of reactions, the reaction products were analyzed in the same manner as in the above (i), whereby it was confirmed that the following dipeptides were formed and accumulated in the respective reaction mixtures: 7.0 g/l L-alanyl-L-phenylalanine (L-Ala-L-Phe) alone; 7.0 g/l L-alanyl-L-methionine (L-Ala-L-Met) and 0.03 g/l L-Ala-L-Ala; 5.0 g/l L-alanyl-L-leucine (L-Ala-L-Leu) and 0.2 g/l L-Ala-L-Ala; and 1.6 g/l L-alanyl-L-valine (L-Ala-L-Val) and 0.3 g/l L-Ala-L-Ala.

(iii) Reactions were carried out under the same conditions as in the above (i) using reaction mixtures having the same composition as that of the reaction mixture of the above (i) except that 0.01 mg of the enzyme was used, Gly was used in place of L-Ala, and L-Phe and L-Met, respectively, were used in place of L-Gln.

After the completion of reactions, the reaction products were analyzed in the same manner as in the above (i), whereby it was confirmed that 5.2 g/l glycyl-L-phenylalanine (Gly-L-Phe) and 1.1 g/l glycyl-L-methionine (Gly-L-Met) were formed and accumulated in the respective reaction mixtures.

When ATP was excluded from the compositions of the above reaction mixtures, no dipeptide was formed.

The above results revealed that the ywfE gene product has the activity to produce, in the presence of ATP, the following dipeptides: L-Ala-L-Gln plus L-Ala-L-Ala, L-Ala-L-Phe, L-Ala-L-Met plus L-Ala-L-Ala, L-Ala-L-Leu plus L-Ala-L-Ala, or L-Ala-L-Val plus L-Ala-L-Ala from L-Ala plus L-Gln, L-Phe, L-Met, L-Leu or L-Val; and Gly-L-Phe or Gly-L-Met from Gly plus L-Phe or L-Met.

### Experimental Example 4

### Production of Dipeptides Using the His-Tagged Recombinant Enzyme (2)

A reaction mixture (0.1 ml) comprising 0.04 mg of the purified His-tagged recombinant enzyme obtained in Experimental Example 2, 100 mmol/l Tris-HCl (pH 8.0), 60 mmol/1 magnesium chloride and 60 mmol/l ATP was prepared. To this mixture were respectively added combinations of various L-amino acids, Gly and β-Ala selected from the amino acids shown in the first row of Table 1 and in the leftmost column of Table 1 to give a concentration of 30 mmol/l each, and the resulting mixtures were subjected to reaction at 37° C. for 16 hours. After the completion of reactions, the reaction products were analyzed by HPLC, whereby it was confirmed that the dipeptides shown in Table 1 were formed.

TABLE 1-1

| | Ala | Gln | Glu | Gly | Val | Leu | Ile | Pro |
|---|---|---|---|---|---|---|---|---|
| Ala | AlaAla | AlaGln | AlaAla | AlaGly | AlaVal | AlaLeu | AlaIle | AlaAla |
| | | AlaAla | | AlaAla | AlaAla | AlaAla | AlaAla | |
| Gln | | x | x | GlyGln | x | x | x | x |
| | | | | GlyGly | | | | |
| Glu | | | | GlyGly | | | | |
| Gly | | | | GlyGly | | | | GlyGly |
| Val | | | | | | | | |
| Leu | | | | | | | | |
| Ile | | | | | | | | |
| Pro | | | | | | | | |
| Phe | | | | | | | | |
| Trp | | | | | | | | |
| Met | | | | | | | | |
| Ser | | | | | | | | |
| Thr | | | | | | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| αAB | | | | | | | | |
| β-Ala | | | | | | | | |
| Cit | | | | | | | | |

TABLE 1-2

| | Phe | Trp | Met | Ser | Thr | Cys | Asn | Tyr |
|---|---|---|---|---|---|---|---|---|
| Ala | AlaPhe | AlaTrp | AlaMet | AlaSer | AlaThr | AlaAla | AlaAsn | AlaTyr |
| | AlaAla | AlaAla | | AlaAla | AlaAla | ∘ | AlaAla | AlaAla |
| Gln | ∘ | x | MetMet | SerGln | ThrGln | ∘ | x | x |
| | | | | SerSer | ThrThr | | | |
| Glu | | | | | | | | |
| Gly | GlyPhe | GlyGly | GlyMet | GlySer | GlyThr | GlyGly | GlyGly | GlyTyr |
| | | ∘ | GlyGly | GlyGly | GlyGly | ∘ | | GlyGly |
| | | | | SerGly | ThrGly | | | |
| | | | | SerSer | ThrThr | | | |
| Val | | | x | | | | | |
| Leu | | | MetMet | | ThrLeu | | | |
| Ile | | | MetMet | | | | | |
| Pro | | | MetMet | SerSer | ThrThr | | | |
| Phe | | | MetPhe | SerPhe | ThrPhe | | | |
| | | | MetMet | | ThrThr | | | |
| Trp | | | | | | | | |
| Met | | | MetMet | SerMet | ThrMet | MetMet | | MetTyr |
| | | | | | ThrThr | ∘ | | MetMet |
| Ser | | | | SerSer | SerThr | | | SerTyr |
| | | | | | SerSer | | | SerSer |
| | | | | | ThrSer | | | |
| | | | | | ThrThr | | | |
| Thr | | | | | ThrThr | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| α AB | | | | | | | | |
| β-Ala | | | | | | | | |
| Cit | | | | | | | | |

TABLE 1-3

| | | | | | | | | Aza- | Thea- |
|---|---|---|---|---|---|---|---|---|---|
| | Lys | Arg | His | Asp | α-AB | β-Ala | Cit | serine | nine |
| Ala | AlaAla | AlaArg | AlaHis | AlaAla | AlaAla | | AlaAla | AlaAla | AlaAla |
| | ∘ | AlaAla | AlaAla | | ∘ | | ∘ | ∘ | ∘ |
| Gln | x | x | x | x | ∘ | | | | |
| Glu | | | | | | | | | |
| Gly | GlyGly | GlyArg | GlyGly | GlyGly | GlyGly | | ∘ | | |
| | ∘ | GlyGly | | | ∘ | | | | |
| Val | | | | | | | | | |
| Leu | | | | | | | | | |
| Ile | | | | | | | | | |
| Pro | | | | | | | | | |
| Phe | | | | x | | ∘ | | | |
| Trp | | | | | | | | | |
| Met | MetMet | | MetMet | | | ∘ | | | |
| | ∘ | | ∘ | | | | | | |
| Ser | | | SerHis | | SerSer | | | | |
| | | | | | ∘ | | | | |
| Thr | | | | | ThrThr | | | | |
| | | | | | ∘ | | | | |
| Cys | | | | | | | | | |
| Asn | | | | | | | | | |
| Tyr | | | | | | | | | |
| Lys | | | | | | | | | |
| Arg | | | | | ∘ | | | | |
| His | | | | | | β-AlaHis | | | |
| Asp | | | | | | | | | |
| α-AB | | | | | ∘ | | | | |
| β-Ala | | | | | | | | | |
| Cit | | | | | | ∘ | | | |

The dipeptides formed by the reaction using, as substrates, two (or one) kinds of L-amino acids, Gly and β-Ala shown in the first row and the leftmost column of Table 1 are shown in the respective cells of the table. In the table, ∘ means that a dipeptide was formed though its sequence was unidentified; x means that formation of a dipeptide was not confirmed; and a blank means that reaction was not carried out.

### Experimental Example 5

### Production of a Dipeptide Using the Strain Expressing the His-Tagged Recombinant Enzyme

Escherichia coli NM522/pQE60ywfE obtained in Experimental Example 1 was inoculated into 8 ml of LB medium containing 50 μg/ml ampicillin in a test tube, and cultured at 28° C. for 17 hours. The resulting culture was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells.

A reaction mixture (20 ml, pH 7.2) comprising 200 g/l wet cells, 50 g/l glucose, 5 g/l phytic acid (diluted to neutrality with 33% conc. sodium hydroxide solution), 15 g/l potassium dihydrogenphosphate, 5 g/l magnesium sulfate heptahydrate, 4 g/l Nymeen S-215, 10 ml/l xylene, 200 mmol/l L-Ala and 200 mmol/l L-Gln was put in a 50-ml beaker, and reaction was carried out at 32° C. at 900 rpm for 2 hours. During the reaction, the pH of the reaction mixture was maintained at 7.2 by using 2 mol/l potassium hydroxide.

The reaction product was analyzed by the same method as in Experimental Example 3, whereby it was confirmed that 25 mg/l L-Ala-L-Gln was accumulated.

### Experimental Example 6

### Cloning of Genes Corresponding to the ywfE Gene from Various Microorganisms of the Genus Bacillus and Analysis Thereof

On the basis of the nucleotide sequence shown in SEQ ID NO: 26, genes corresponding to the ywfE gene which exist in Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were obtained in the following manner.

That is, Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were respectively inoculated into LB medium and subjected to static culture overnight at 30° C. After the culturing, the chromosomal DNAs of the respective microorganisms were isolated and purified according to the method using saturated phenol described in Current Protocols in Molecular Biology.

By using a DNA synthesizer (Model 8905, PerSeptive Biosystems, Inc.), DNAs having the nucleotide sequences shown in SEQ ID NOS: 37 and 38 (hereinafter referred to as primer C and primer D, respectively) were synthesized. Primer C has a sequence containing a region upstream of the initiation codon of ywfE gene of the chromosomal DNA of Bacillus subtilis 168, and primer D has a sequence complementary to a sequence containing a region downstream of the termination codon of ywfE gene.

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and the above primer C and primer D as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

One-tenth of each of the resulting reaction mixtures was subjected to agarose gel electrophoresis to confirm that a ca. 1.4 kb fragment corresponding to the ywfE gene fragment was amplified. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained 1.4 kb DNA fragments derived from the chromosomal DNAs of the respective strains and pCR-blunt (Invitrogen Corp.) were subjected to ligation reaction using a ligation kit at 16° C. for 16 hours.

Escherichia coli NM522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following plasmids containing a gene corresponding to the ywfE gene were obtained: pYWFE1 (derived from ATCC 15245, DNA having the nucleotide sequence shown in SEQ ID NO: 65), pYWFE2 (derived from ATCC 6633, DNA having the nucleotide sequence shown in SEQ ID NO: 27), pYWFE3 (derived from IAM 1213, DNA having the nucleotide sequence shown in SEQ ID NO: 28), pYWFE4 (derived from IAM 1107, DNA having the nucleotide sequence shown in SEQ ID NO: 29), pYWFE5 (derived from IAM 1214, DNA having the nucleotide sequence shown in SEQ ID NO: 30), pYWFE6 (derived from ATCC 9466, DNA having the nucleotide sequence shown in SEQ ID NO: 26), pYWFE7 (derived from IAM 1033, DNA having the nucleotide sequence shown in SEQ ID NO: 65), pYWFE8 (derived from ATCC 21555, DNA having the nucleotide sequence shown in SEQ ID NO: 31) and pYWFE9 (derived from IFO 3022, DNA having the nucleotide sequence shown in SEQ ID NO: 32).

On the other hand, a gene corresponding to ywfE gene derived from Bacillus pumilus NRRL B-12025 (DNA having the nucleotide sequence shown in SEQ ID NO: 64) was obtained in the following manner.

PCR was carried out using the chromosomal DNA of the NRRL B-12025 strain prepared above as a template and DNAs respectively consisting of the nucleotide sequences shown in SEQ ID NOS: 66 and 67 as a set of primers. That is, PCR was carried out by 30 cycles, one cycle consisting of reaction at 98° C. for 5 seconds, reaction at 55° C. for 30 seconds and reaction at 72° C. for one minute, using 50 μl of a reaction mixture comprising 0.1 μg of the chromosomal DNA, 0.5 μmol/l each of the primers, 2.5 units of Z-taq polymerase (Takara Bio Inc.), 5 μl of buffer for Z-taq polymerase (10×) (Takara Bio Inc.) and 200 μmol/l each of dNTPs.

One-tenth of the resulting reaction mixture was subjected to agarose gel electrophoresis to confirm that a ca. 0.8 kb fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained 0.8 kb fragment derived from the chromosomal DNA and pGEM T-easy (Promega Corp.) were subjected to ligation reaction using a ligation kit at 16° C. for 16 hours.

Escherichia coli DH5α was transformed using the reaction mixture according to the method using calcium ion, spread on LB agar medium containing 50 μg/ml ampicillin, and cultured overnight at 30° C.

A plasmid was extracted from the transformant obtained above and the nucleotide sequence of the ca. 0.8 kb DNA insert was determined, whereby a sequence from nucleotides 358 to 1160 in the nucleotide sequence shown in SEQ ID NO: 64 was confirmed.

The above plasmid was cleaved with EcoRI and then subjected to agarose gel electrophoresis to separate a DNA fragment. The DNA fragment was purified using GENECLEAN II Kit, and about 0.5 μg of the purified DNA fragment was DIG-labeled using DIG-High Prime DNA Labeling & Detection Starter Kit I (Roche Diagnostics Corp.) according to the instructions attached thereto.

Southern analysis of the chromosomal DNA of the NRRL B-12025 strain was carried out using the DIG-labeled DNA obtained above.

The chromosomal DNA of the NRRL B-12025 strain was completely digested with BamHI, EcoRI, HindIII, KpnI, PstI, SacI, SalI and SphI, respectively, and subjected to agarose gel electrophoresis to separate DNA fragments, followed by transfer to nylon membrane plus charge (Roche Diagnostics Corp.) according to an ordinary method.

After the DNA fragments were fixed on the nylon membrane by UV irradiation, Southern hybridization was carried out using the above probe DNA and the nylon membrane.

The hybridization was carried out by contacting the nylon membrane with the probe DNA at 65° C. for 16 hours, washing the nylon membrane twice with a solution consisting of 0.1% SDS and 2×SSC at room temperature for 5 minutes, and further washing the membrane twice with a solution consisting of 0.1% SDS and 0.5×SSC at 65° C. for 15 minutes. The other operations and conditions and detection of the hybridized DNA were carried out according to the instructions attached to the above-mentioned DIG-High Prime DNA Labeling & Detection Starter Kit I.

As a result, color development was observed at around 3.5 kbp of the fragments completely digested with HindIII and PstI.

Subsequently, the chromosomal DNA of the NRRL B-12025 strain was completely digested with HindIII and PstI, respectively, and subjected to agarose gel electrophoresis to separate DNA fragments. From the respective restriction enzyme-digested DNAs, 3-4 kbp fragments were purified using GENECLEAN II Kit, followed by autocyclization using a ligation kit.

On the basis of the nucleotide sequence of the 0.8 kb DNA fragment determined above, the nucleotide sequences shown in SEQ ID NOS: 71 and 72 were designed and synthesized, and they were used in PCR using the cyclized DNA obtained above as a template. PCR was carried out by 30 cycles, one cycle consisting of reaction at 98° C. for 5 seconds, reaction at 55° C. for 30 seconds and reaction at 72° C. for 3 minutes and 30 seconds, using 50 μl of a reaction mixture comprising 10 ng of the cyclized DNA, 0.5 μmol/l each of the primers, 2.5 units of pyrobest polymerase (Takara Bio Inc.), 5 μl of buffer for pyrobest polymerase (10×) (Takara Bio Inc.) and 200 μmol/l each of dNTPs.

One-tenth of the resulting reaction mixture was subjected to agarose gel electrophoresis to confirm that a ca. 3.0 kb fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained DNA fragment and Zero Blunt PCR Cloning Kit (Invitrogen Corp.) were subjected to ligation reaction using a ligation kit.

Escherichia coli NM522 was transformed using the reaction mixture according to the method using calcium ion, spread on LB agar medium containing 50 μg/ml ampicillin, and cultured overnight at 30° C.

A plasmid was extracted from a colony of the transformant that grew on the medium according to a known method and the structure of the plasmid was analyzed using restriction enzymes. As a result, it was confirmed that plasmid pYWFE10 (derived from NRRL B-12025, DNA having the nucleotide sequence shown in SEQ ID NO: 64) containing a gene corresponding to the ywfE gene was obtained.

The nucleotide sequences of the genes corresponding to the ywfE gene which are respectively contained in the plasmids pYWFE1 to pYWFE10 obtained above were determined using 373A DNA Sequencer.

The amino acid sequences of the proteins encoded by the genes respectively contained in pYWFE1, pYWFE6 and pYWFE7 were identical with the amino acid sequence of the protein encoded by the ywfE gene, whereas those of the proteins encoded by the genes respectively contained in pYWFE2, pYWFE3, pYWFE4, pYWFE5, pYWFE8, pYWFE9 and pYWFE10 were different from the amino acid sequence of the protein encoded by the ywfE gene.

The amino acid sequences of the proteins encoded by the genes corresponding to the ywfE gene which are contained in pYWFE2, pYWFE3, pYWFE4, pYWFE5, pYWFE8, pYWFE9 and pYWFE10, and pYWFE1 and pYWFE7 are shown in SEQ ID NOS: 20 to 25, 68 and 19, respectively, and the nucleotide sequences of these genes are shown in SEQ ID NOS: 27 to 32, 65 and 26, respectively.

### Experimental Example 7

### Purification of C-Terminal His-Tagged Recombinant Dipeptide Synthetase

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and primer A and primer B described in Experimental Example 1 as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

When the chromosomal DNA of Bacillus pumilus NRRL B-12025 was used as a template, PCR was carried out using DNAs respectively having the nucleotide sequences shown in SEQ ID NOS: 69 and 70 as a set of primers under the same conditions as above.

One-tenth of each of the resulting reaction mixtures was subjected to agarose gel electrophoresis to confirm that a ca. 1.4 kb DNA fragment corresponding to the ywfE fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained solutions (5 μl) was subjected to reaction to cleave the amplified DNA with restriction enzymes NcoI and BamHI. DNA fragments were separated by agarose gel electrophoresis, and a 1.4 kb DNA fragment containing a gene corresponding to the ywfE gene was recovered using GENECLEAN II Kit.

Subsequently, 0.2 μg of the C-terminal His-tagged recombinant expression vector pQE60 was cleaved with restriction enzymes NcoI and BamHI. DNA fragments were separated by agarose gel electrophoresis, and a 3.4 kb DNA fragment was recovered in the same manner as above.

Each of the 1.4 kb DNA fragments containing a gene corresponding to the ywfE gene of Bacillus subtilis 168 and the 3.4 kb DNA fragment obtained above were subjected to ligation reaction using a ligation kit at 16° C. for 16 hours. Escherichia coli NM522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following C-terminal His-tagged gene expression vectors were obtained: pQE60ywfE1 (a vector containing the gene derived from ATCC 15245), pQE60ywfE2 (a vector containing the gene derived from ATCC 6633), pQE60ywfE3 (a vector containing the gene derived from IAM 1213), pQE60ywfE4 (a vector containing the gene derived from IAM 1107), pQE60ywfE5 (a vector containing the gene derived from IAM 1214), pQE60ywfE6 (a vector containing the gene derived from ATCC 9466), pQE60ywfE7 (a vector containing the gene derived from IAM 1033), pQE60ywfE8 (a vector containing the gene derived from ATCC 21555), pQE60ywfE9 (a vector containing the gene derived from IFO 3022) and pQE60ywfE10 (a vector containing the gene derived from NRRL B-12025).

Escherichia coli NM522/pQE60ywfE1 to NM522/pQE60ywfE10 strains obtained above were respectively inoculated into 8 ml of LB medium containing 50 μg/ml ampicillin in a test tube, and cultured at 28° C. for 17 hours. Each of the resulting cultures was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells, and His-tagged recombinant enzymes were purified from the respective wet cells using H isTrap according to the instructions attached thereto.

### Experimental Example 8

### Production of Dipeptides Using Purified Enzymes

Reaction mixtures (0.1 ml each) comprising 0.04 mg of the respective recombinant enzymes obtained in Experimental Example 7, 100 mmol/l Tris-HCl (pH 8.0), 60 mmol/1 magnesium chloride, 60 mmol/l ATP, 30 mmol/l L-Ala and 30 mmol/l L-Gln were prepared, and reactions were carried out at 37° C. for 16 hours.

After the completion of reactions, the reaction mixtures were analyzed by the method described in Experimental Example 3, whereby it was confirmed that 3.0 to 3.5 g/l L-Ala-L-Gln and 0.25 to 0.3 g/l L-Ala-L-Ala were formed and accumulated.

When ATP was excluded from the compositions of the above reaction mixtures, L-Ala-L-Gln or L-Ala-L-Ala was not formed at all.

The above results revealed that all of the products of the genes obtained in Experimental Example 7 have the activity to produce L-Ala-L-Gln and L-Ala-L-Ala from L-Ala and L-Gln in the presence of ATP.

### Experimental Example 9

### Construction of Escherichia coli for Enhanced Expression of the ywfE Gene

By using a DNA synthesizer (Model 8905, PerSeptive Biosystems, Inc.), DNAs having the sequences shown in SEQ ID NOS: 60 to 63 (hereinafter referred to as primer E, primer F, primer G and primer H, respectively) were synthesized. The sequence of SEQ ID NO: 60 is a sequence wherein a sequence containing the XhoI recognition sequence is added to the 5′ end of a region containing the Shine-Dalgarno sequence (ribosome binding sequence) of ywfE gene on the plasmid pQE60ywfE. The sequence of SEQ ID NO: 61 is a sequence wherein a sequence containing the BamHI recognition sequence is added to the 5′ end of a sequence complementary to a sequence containing the termination codon of ywfE gene. The sequence of SEQ ID NO: 62 is a sequence wherein a sequence containing the EcoRI recognition sequence is added to the 5′ end of the sequence of trp promoter region of expression vector pTrS30 containing trp promoter. The sequence of SEQ ID NO: 63 is a sequence wherein a sequence containing the XhoI recognition sequence is added to the 5′ end of a sequence complementary to the sequence of trp promoter region of expression vector pTrS30 containing trp promoter.

A ywfE gene fragment and a trp promoter region fragment were amplified by PCR using the above primers E and F and primers G and H as a set of primers, respectively, and the plasmid pQE60ywfE as a template. PCR was carried out by 30 cycles, one cycle consisting of reaction at 94° C. for one minute, reaction at 55° C. for 2 minutes and reaction at 72° C. for 3 minutes, using 40 μl of a reaction mixture comprising 10 ng of pQE60ywfE, 0.5 μmol/l each of the primers, 2.5 units of Pfu DNA polymerase, 4 μl of buffer for Pfu DNA polymerase (10×) and 200 μmol/l each of dNTPs.

One-tenth of each of the resulting reaction mixtures was subjected to agarose gel electrophoresis to confirm that a ca. 1.4 kb fragment corresponding to the ywfE gene fragment and a ca. 0.3 kb fragment corresponding to the trp promoter region fragment were respectively amplified in the PCR using primer E and primer F and the PCR using primer G and primer H. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA was dissolved in 20 μl of TE.

The thus obtained DNA solutions (5 μl each) were respectively subjected to reaction to cleave the DNA amplified using primer E and primer F with restriction enzymes XhoI and BamHI and to reaction to cleave the DNA amplified using primer G and primer H with restriction enzymes EcoRI and XhoI. DNA fragments were separated by agarose gel electrophoresis, and a 1.4 kb fragment containing ywfE gene and a 0.3 kb fragment containing trp promoter region were respectively recovered using GENECLEAN II Kit.

pTrs30 (a trp promoter-containing expression vector, 0.2 μg) was cleaved with restriction enzymes EcoRI and BamHI. DNA fragments were separated by agarose gel electrophoresis and a 4.5 kb DNA fragment was recovered in the same manner as above.

The 1.4 kb fragment containing the ywfE gene, the 0.3 kb fragment containing trp promoter region and the 4.5 kb DNA fragment obtained above were subjected to ligation reaction using a ligation kit at 16° C. for 16 hours.

Escherichia coli NM522 was transformed using the reaction mixture according to the method using calcium ion, spread on LB agar medium containing 50 μg/ml ampicillin, and cultured overnight at 30° C.

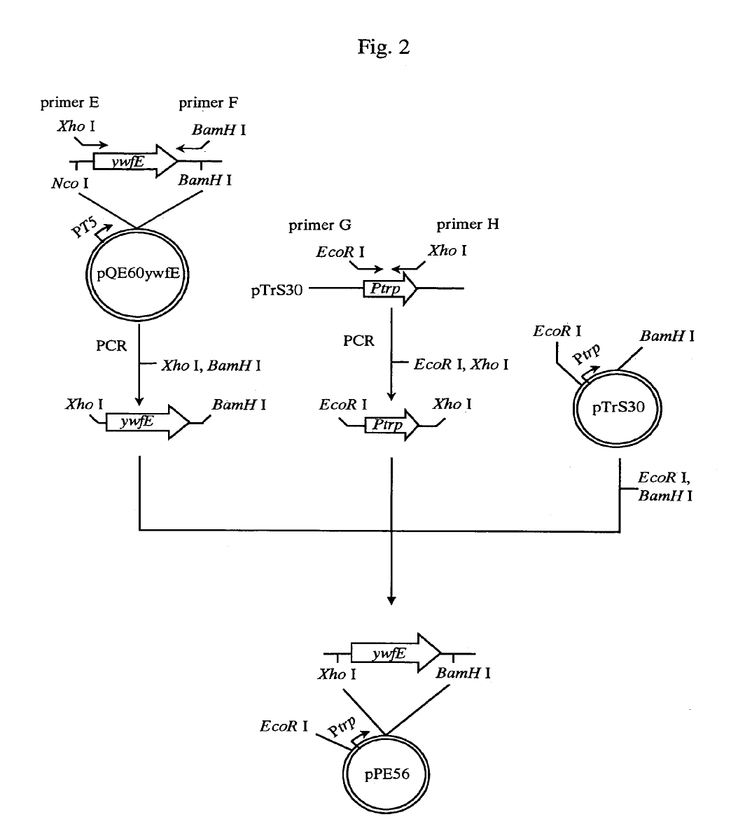

A plasmid was extracted from a colony of the transformant that grew on the medium according to a known method, whereby expression vector pPE56 containing the ywfE gene in a downstream position of the trp promoter was obtained. The structure of the vector was confirmed by digestion with restriction enzymes (FIG. 2).

Certain embodiments of the present invention are illustrated in the following examples. These examples are not to be construed as limiting the scope of the invention.

### Experimental Example 10

### Preparation of Strains Having pepD Gene, pepN Gene, pepB Gene, pepA Gene and dpp Operon Deletions

Strains in which specific genes on Escherichia coli chromosomal DNA are deleted were prepared according to the method utilizing the homologous recombination system of lambda phage [Proc. Natl. Acad. Sci. USA, 97, 6641-6645 (2000)].

Plasmids pKD46, pKD3 and pCP20 used below were prepared by extraction from Escherichia coli strains carrying them which were obtained from Escherichia coli Genetic Stock Center, Yale University, U.S.A.

### (1) Cloning of DNA Fragments for Gene Deletion

For the purpose of deleting the following genes existing on the chromosomal DNA of Escherichia coli K12, DNAs having nucleotide sequences homologous to 36-bp nucleotide sequences that lie upstream and downstream of the respective genes to be deleted on the chromosomal DNA of Escherichia coli K12 and the nucleotide sequence shown in SEQ ID NO: 39 which is recognized by yeast-derived Flp recombinase were synthesized using a DNA synthesizer (Model 8905, PerSeptive Biosystems, Inc.). The genes to be deleted are pepD gene having the nucleotide sequence shown in SEQ ID NO: 10, pepN gene having the nucleotide sequence shown in SEQ ID NO: 11, pepB gene having the nucleotide sequence shown in SEQ ID NO: 12, pepA gene having the nucleotide sequence shown in SEQ ID NO: 13, dppA gene having the nucleotide sequence shown in SEQ ID NO: 14, dppB gene having the nucleotide sequence shown in SEQ ID NO: 15, dppC gene having the nucleotide sequence shown in SEQ ID NO: 16, dppD gene having the nucleotide sequence shown in SEQ ID NO: 17 and dppF gene having the nucleotide sequence shown in SEQ ID NO: 18. In the case of dppA gene, dppB gene, dppC gene, dppD gene and dppF gene, which form an operon, DNAs having nucleotide sequences homologous to the nucleotide sequences that lie upstream and downstream of the operon were synthesized.

That is, DNAs consisting of the following nucleotide sequences were synthesized as respective sets of primers for amplification of DNA fragments for gene deletion: SEQ ID NOS: 40 and 41 for pepD gene deletion; SEQ ID NOS: 42 and 43 for pepN gene deletion; SEQ ID NOS: 44 and 45 for pepA gene deletion; SEQ ID NOS: 46 and 47 for pepB gene deletion; and SEQ ID NOS: 48 and 49 for dpp operon deletion.

Subsequently, PCR was carried out using each set of the above synthetic DNAs as a set of primers and pKD3 DNA as a template. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 10 ng of the plasmid DNA, 0.5 μmol/l each of the primers, 2.5 units of Pfu DNA polymerase (Stratagene), 4 μl of buffer for Pfu DNA polymerase (10×) (Stratagene) and 200 μmol/l each of deoxyNTPs (dATP, dGTP, dCTP and dTTP).

One-tenth of each of the resulting reaction mixtures was subjected to agarose gel electrophoresis to confirm that the desired fragment was amplified. Then, the remaining reaction mixture was mixed with an equal amount of phenol/chloroform (1 vol/1 vol) saturated with TE [10 mmol/l Tris-HCl (pH 8.0), 1 mmol/l EDTA].

The resulting mixture was centrifuged, and the obtained upper layer was mixed with a two-fold volume of cold ethanol and allowed to stand at −80° C. for 30 minutes, followed by centrifugation. By this procedure, chloramphenicol resistance gene-containing DNA fragments for deletion of pepD gene, pepN gene, pepB gene, pepA gene and dpp operon were obtained.

(2) Preparation of Escherichia coli JM101 Having pepD Gene Deletion

Escherichia coli JM101 was transformed with pKD46, spread on LB agar medium containing 100 mg/l ampicillin, and cultured at 30° C. to select a transformant.

The plasmid pKD46 carries inserted λ Red recombinase gene and is designed so that the expression of the gene is induced by L-arabinose. Accordingly, when the Escherichia coli grown in the presence of L-arabinose is transformed using a linear DNA, homologous recombination occurs with high frequency. Further, as pKD46 has a thermosensitive replication origin, curing of the plasmid can be readily caused by culturing the strain at 42° C.

The chloramphenicol resistance gene-containing DNA fragment for pepD gene deletion obtained above was introduced into Escherichia coli JM101/pKD46 obtained by culturing in the presence of 10 mmol/l L-arabinose and 50 μg/ml ampicillin by electroporation. The resulting cells were spread on LB agar medium (10 g/l Bacto-tryptone, 5 g/l Bacto-yeast extract, 5 g/l sodium chloride and 15 g/l agar) containing 25 mg/l chloramphenicol and cultured at 30° C. to select a transformant in which the chloramphenicol resistance gene-containing DNA fragment for pepD gene deletion was integrated into the chromosomal DNA of Escherichia coli JM101 by homologous recombination.

The selected chloramphenicol-resistant strain was inoculated onto LB agar medium containing 25 mg/l chloramphenicol and cultured at 42° C. for 14 hours, followed by single colony isolation. Replicas of the obtained colonies were made on LB agar medium containing 25 mg/l chloramphenicol and LB agar medium containing 100 mg/l ampicillin, followed by culturing at 37° C. By selecting a colony showing chloramphenicol resistance and ampicillin sensitivity, a pKD46-cured strain was obtained.

The pKD46-cured strain thus obtained was transformed using pCP20, followed by selection on LB agar medium containing 100 mg/l ampicillin to obtain a pKD46-cured strain carrying pCP20.

The plasmid pCP20 carries inserted yeast-derived Flp recombinase gene and is designed so that the expression of the gene is induced at a temperature of 42° C.

The chloramphenicol resistance gene-containing DNA fragments for deletion of pepD gene, pepN gene, pepB gene, pepA gene and dpp operon prepared above contain nucleotide sequences recognized by Flp recombinase at both termini of the chloramphenicol resistance gene. Therefore, the resistance gene can be readily deleted by homologous recombination catalyzed by Flp recombinase.

Further, as pCP20 has a thermosensitive replication origin, expression of Flp recombinase and curing of pCP20 can be simultaneously induced by culturing the pCP20-carrying strain at 42° C.

The pCP20-carrying pKD46-cured strain obtained above was inoculated onto drug-free LB agar medium and cultured at 42° C. for 14 hours, followed by single colony isolation. Replicas of the obtained colonies were made on drug-free LB agar medium, LB agar medium containing 25 mg/l chloramphenicol and LB agar medium containing 100 mg/l ampicillin, followed by culturing at 30° C. Then, colonies showing chloramphenicol sensitivity and ampicillin sensitivity were selected.

Chromosomal DNAs were prepared from the respective strains selected above according to an ordinary method [Seibutsukogaku Jikkensho (Experiments in Biotechnology), edited by The Society for Biotechnology, Japan, p. 97-98, Baifukan (1992)]. PCR was carried out using, as a set of primers, DNAs having the nucleotide sequences shown in SEQ ID NOS: 50 and 51 which were designed based on an inner nucleotide sequence of the pepD gene to be deleted, and using each of the chromosomal DNAs as a template. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of deoxyNTPs.

A strain with which no amplified DNA fragment was detected in the above PCR was identified as a strain having pepD gene deletion and was designated as Escherichia coli JPD1.

(3) Preparation of a Strain in Which the pepD Gene and pepN Gene on the Chromosomal DNA of Escherichia coli JM101 are Deleted

Escherichia coli JPD1 obtained in the above (2) was transformed with pKD46, spread on LB agar medium containing 100 mg/l ampicillin, and cultured at 30° C. to select a transformant. The chloramphenicol resistance gene-containing DNA fragment for pepN gene deletion was introduced into the obtained transformant (Escherichia coli JPD1/pKD46) by electroporation to obtain a transformant in which the chloramphenicol resistance gene-containing DNA fragment for pepN gene deletion was integrated into the chromosomal DNA of Escherichia coli JPD1/pKD46 by homologous recombination.

Subsequently, the same procedure as in the above (2) was carried out to obtain a strain in which the chloramphenicol resistance gene was deleted from the chromosomal DNA, which was designated as Escherichia coli JPDN2.

(4) Preparation of Strains in which pepN Gene, pepA Gene, pepB Gene or dpp Operon on the Chromosomal DNA of Escherichia coli JM101 is Deleted and Strains Having Multiple Gene Deletion

The strains having pepN gene, pepA gene, pepB gene or dpp operon deletion were prepared according to the same procedure as in the above (2) using the respective chloramphenicol resistance gene-containing DNA fragments for gene or operon deletion prepared in the above (1).

Acquisition of the strains having gene deletions by the above method was confirmed by carrying out PCR in the same manner as in the above (2) using, as sets of primers, DNAs having the nucleotide sequences shown in SEQ ID NOS: 52 to 59 which were designed and synthesized based on inner nucleotide sequences of the respective genes to be deleted. That is, DNAs having the following nucleotide sequences were used as respective sets of primers for the confirmation of gene deletion: SEQ ID NOS: 52 and 53 for pepN gene deletion; SEQ ID NOS: 54 and 55 for pepA gene deletion; SEQ ID NOS: 56 and 57 for pepB gene deletion; and SEQ ID NOS: 58 and 59 for dpp operon deletion.

The thus obtained dpp operon-deleted strain, pepN gene-deleted strain, pepA gene-deleted strain and pepB gene-deleted strain were designated as Escherichia coli JDPP1, Escherichia coli JPN1, Escherichia coli JPA1 and Escherichia coli JPB7, respectively.

Further, strains having multiple gene deletions, i.e., deletions of two or more genes or operon selected from the group consisting of pepD gene, pepN gene, pepA gene, pepB gene and dpp operon were prepared according to the method of the above (3). Acquisition of the strains having multiple gene deletions was confirmed by PCR similar to that in the above (2). The thus obtained double gene-deleted strain having pepD gene and dpp operon deletions was designated as Escherichia coli JPDP49, triple gene-deleted strain having pepB gene, pepD gene and pepN gene deletions as Escherichia coli JPDNB43, triple gene-deleted strain having pepD gene, pepN gene and dpp operon deletions as Escherichia coli JPNDDP36, quadruple gene-deleted strain having pepA gene, pepD gene, pepN gene and dpp operon deletions as Escherichia coli JPNDAP5, and quadruple gene-deleted strain having pepB gene, pepD gene, pepN gene and dpp operon deletions as Escherichia coli JPNDBP7. The genes deleted in the gene-deleted strains are shown in Table 2.

TABLE 2

| Strain | Deleted gene |
|---|---|
| JM101 | none |
| JDPP1 | dpp operon |
| JPN1 | pepN |
| JPA1 | pepA |
| JPB7 | pepB |
| JPD1 | pepD |
| JPDN2 | pepD, pepN |
| JPNDB43 | pepB, pepD, pepN |
| JPDP49 | pepD, dpp operon |
| JPNDDP36 | pepD, pepN, dpp operon |
| JPNDAP5 | pepA, pepD, pepN, dpp operon |
| JPNDBP7 | pepB, pepD, pepN, dpp operon |

### Experimental Example 11

### Evaluation of Productivity of L-Alanyl-L-glutamine (Hereinafter Referred to as AlaGln) and L-Alanyl-L-alanine (Hereinafter Referred to as AlaAla) by Escherichia coli Strains in which Peptidase and Peptide-Transporting Activities are Lost

The strains having deletions of genes encoding various peptidase genes and an operon encoding peptide-transporting protein which were obtained in Example 1 were transformed using the plasmid pPE56 constructed in Experimental Example 8 to obtain ampicillin-resistant transformants.

Each of the obtained transformants was inoculated into 8 ml of LB medium containing 50 μg/ml ampicillin in a test tube and cultured at 28° C. for 17 hours. The resulting culture was inoculated into 8 ml of a production medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l L-Ala, 2.5 g/l L-Gln, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution; L-Gln was added after sterilization by filtration of a 10-fold conc. solution; glucose, vitamin B₁, magnesium sulfate heptahydrate and ferrous sulfate heptahydrate were added after separate autoclaving] containing 100 μg/ml ampicillin in a test tube in an amount of 1% and cultured at 30° C. for 24 hours. The resulting culture was centrifuged to obtain a culture supernatant.

The product in the culture supernatant was derivatized by the F-moc method and then analyzed by HPLC. The HPLC analysis was carried out using ODS-HG5 (Nomura Kagaku Co., Ltd.) as a separation column and solution A (6 ml/l acetic acid and 20% (v/v) acetonitrile, pH adjusted to 4.8 with triethylamine) and solution B (6 ml/l acetic acid and 70% (v/v) acetonitrile, pH adjusted to 4.8 with triethylamine) as eluting solutions. The ratio of solution A to solution B was 8:2 during the first 5 minutes of elution and thereafter changed with a linear gradient so that the ratio became 1:1 at 20 minutes after the start of elution. The results of analysis are shown in Table 3.

TABLE 3

| | | AlaGln | AlaAla |
|---|---|---|---|
| Strain | Deleted gene | (g/l) | (g/l) |
| JM101 | none | 0 | 0 |
| JDPP1 | dpp operon | 0.02 | 0.01 |
| JPN1 | pepN | 0.01 | 0.01 |
| JPA1 | pepA | 0.01 | 0.01 |
| JPB7 | pepB | 0.01 | 0.01 |
| JPD1 | pepD | 0.01 | 0.01 |
| JPDN2 | pepD, pepN | 0.02 | 0.03 |
| JPNDB43 | pepB, pepD, pepN | 0.05 | 0.12 |
| JPDP49 | pepD, dpp operon | 0.11 | 0.08 |
| JPNDDP36 | pepD, pepN, dpp operon | 0.16 | 0.21 |
| JPNDAP5 | pepA, pepD, pepN, dpp operon | 0.28 | 0.26 |
| JPNDBP7 | pepB, pepD, pepN, dpp operon | 0.43 | 0.22 |

As can be seen from Table 3, small amounts of dipeptides were formed and accumulated by use of the microorganisms having deletions of two or less kinds of peptidase genes or one kind of peptide-transporting protein gene, whereas the amounts of dipeptides formed and accumulated were greatly increased by use of the microorganisms having deletions of one or more kinds of peptidase genes and one kind of peptide-transporting protein gene or microorganisms having deletions of three or more kinds of peptidase genes.

### Experimental Example 12

### Evaluation of Productivity of L-Alanyl-L-valine (Hereinafter Referred to as AlaVal) by Escherichia coli Strains in which Peptidase and Peptide-Transporting Protein Activities are Lost

Similarly to Example 2, the Escherichia coli strains having deletions of various peptidase genes and an operon encoding a peptide-transporting protein were transformed using pPE56. Each of the obtained transformants was inoculated into 8 ml of LB medium containing 50 μg/ml ampicillin in a test tube and cultured at 28° C. for 17 hours. The resulting culture was inoculated into 8 ml of a production medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l L-Ala, 2.5 g/l L-Val, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution; glucose, vitamin B₁, magnesium sulfate heptahydrate and ferrous sulfate heptahydrate were added after separate autoclaving] containing 100 μg/ml ampicillin in a test tube in an amount of 1% and cultured at 30° C. for 24 hours. The resulting culture was centrifuged to obtain a culture supernatant.

The product in the culture supernatant was analyzed by the method described in Example 2. The results are shown in Table 4.

TABLE 4

| | | AlaVal |
|---|---|---|
| Strain | Deleted gene | (g/l) |
| JM101 | none | 0 |
| JDPP1 | dpp operon | 0 |
| JPN1 | pepN | 0 |
| JPA1 | pepA | 0 |
| JPB7 | pepB | 0 |
| JPD1 | pepD | 0 |
| JPDN2 | pepD, pepN | 0 |
| JPNDB43 | pepB, pepD, pepN | 0.04 |
| JPDP49 | pepD, dpp operon | 0.11 |
| JPNDDP36 | pepD, pepN, dpp operon | 0.22 |
| JPNDBP7 | pepB, pepD, pepN, dpp operon | 0.20 |

As can be seen from Table 4, the dipeptide was not produced by use of the microorganisms having deletions of two or less kinds of peptidase genes or one kind of peptide-transporting protein gene, whereas the dipeptide was produced by use of the microorganisms having deletions of three or more kinds of peptidase genes or microorganisms having deletions of one or more kinds of peptidase genes and one kind of peptide-transporting protein gene.

### Experimental Example 13

### Evaluation of Productivity of Glycyl-L-glutamine (Hereinafter Referred to as GlyGln) by Escherichia coli Strains in which Peptidase and Peptide-Transporting Protein Activities are Lost

Similarly to Example 2, the strains having deletions of various peptidase genes and an operon encoding a peptide-transporting protein were transformed using pPE56. Each of the obtained transformants was inoculated into 8 ml of LB medium containing 50 μg/ml ampicillin in a test tube and cultured at 28° C. for 17 hours.

The resulting culture was inoculated into 8 ml of a production medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l Gly, 2.5 g/l L-Gln, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution; L-Gln was added after sterilization by filtration of a 10-fold conc. solution; glucose, vitamin B₁, magnesium sulfate heptahydrate and ferrous sulfate heptahydrate were added after separate autoclaving] containing 100 μg/ml ampicillin in a test tube in an amount of 1% and cultured at 30° C. for 24 hours. The resulting culture was centrifuged to obtain a culture supernatant.

The product in the culture supernatant was analyzed by the method described in Example 2. The results are shown in Table 5.

TABLE 5

| | | GlyGln |
|---|---|---|
| Strain | Deleted gene | (g/l) |
| JM101 | none | 0 |
| JDPP1 | dpp operon | 0 |
| JPDN2 | pepD, pepN | 0 |
| JPNDB43 | pepB, pepD, pepN | 0.01 |
| JPNDDP36 | pepD, pepN, dpp operon | 0.02 |
| JPNDBP7 | pepB, pepD, pepN, dpp operon | 0.03 |

As can be seen from Table 5, the dipeptide was not produced by use of the microorganisms having deletions of two or less kinds of peptidase genes or one kind of peptide-transporting protein gene, whereas the dipeptide was produced by use of the microorganisms having deletions of three or more kinds of peptidase genes or microorganisms having deletions of two or more kinds of peptidase genes and one kind of peptide-transporting protein gene.

### Sequence Listing Free Text

### SEQ ID NO: 35—Description of Artificial Sequence: Synthetic DNA

### SEQ ID NO: 36—Description of Artificial Sequence: Synthetic DNA

### SEQ ID NO: 37—Description of Artificial Sequence: Synthetic DNA

### SEQ ID NO: 38—Description of Artificial Sequence: Synthetic DNA

### SEQ ID NO: 39—Description of Artificial Sequence: Synthetic DNA

### SEQ ID NO: 40—Description of Artificial Sequence: Synthetic DNA

### SEQ ID NO: 41—Description of Artificial Sequence: Synthetic DNA

### SEQ ID NO: 42—Description of Artificial Sequence: Synthetic DNA

### SEQ ID NO: 43—Description of Artificial Sequence: Synthetic DNA

### SEQ ID NO: 44—Description of Artificial Sequence: Synthetic DNA

### SEQ ID NO: 45—Description of Artificial Sequence: Synthetic DNA

### SEQ ID NO: 46—Description of Artificial Sequence: Synthetic DNA

### SEQ ID NO: 47—Description of Artificial Sequence: Synthetic DNA

### SEQ ID NO: 48—Description of Artificial Sequence: Synthetic DNA

### SEQ ID NO: 49—Description of Artificial Sequence: Synthetic DNA

### SEQ ID NO: 50—Description of Artificial Sequence: Synthetic DNA

### SEQ ID NO: 51—Description of Artificial Sequence: Synthetic DNA

### SEQ ID NO: 52—Description of Artificial Sequence: Synthetic DNA

### SEQ ID NO: 53—Description of Artificial Sequence: Synthetic DNA

### SEQ ID NO: 54—Description of Artificial Sequence: Synthetic DNA

### SEQ ID NO: 55—Description of Artificial Sequence: Synthetic DNA

### SEQ ID NO: 56—Description of Artificial Sequence: Synthetic DNA

### SEQ ID NO: 57—Description of Artificial Sequence: Synthetic DNA

### SEQ ID NO: 58—Description of Artificial Sequence: Synthetic DNA

### SEQ ID NO: 59—Description of Artificial Sequence: Synthetic DNA

### SEQ ID NO: 60—Description of Artificial Sequence: Synthetic DNA

### SEQ ID NO: 61—Description of Artificial Sequence: Synthetic DNA

### SEQ ID NO: 62—Description of Artificial Sequence: Synthetic DNA

### SEQ ID NO: 63—Description of Artificial Sequence: Synthetic DNA

### SEQ ID NO: 66—Description of Artificial Sequence: Synthetic DNA

### SEQ ID NO: 67—Description of Artificial Sequence: Synthetic DNA

### SEQ ID NO: 69—Description of Artificial Sequence: Synthetic DNA

### SEQ ID NO: 70—Description of Artificial Sequence: Synthetic DNA

### SEQ ID NO: 71—Description of Artificial Sequence: Synthetic DNA

SEQ ID NO: 72—Description of Artificial Sequence: Synthetic DNA

1-21. (canceled)

22. An isolated microorganism, belonging to the genus Escherichia, Bacillus, Corynebacterium or Saccharomyces, in which (i) a peptidase activity is eliminated, said peptidase activity normally being provided by a protein comprising the amino acid sequence of SEQ ID NO:3, or a protein having an amino acid sequence which has 95% or more homology to SEQ ID NO:3 and having peptidase activity, (ii) a peptide transport across membrane activity is eliminated, said peptide transport across membrane activity normally being provided by a protein complex comprising the amino acid sequence of SEQ ID NOS:5 to 9, or a protein complex comprising proteins having an amino acid sequence which has 95% or more homology to SEQ ID NOS:5 to 9 and having peptide transport activity; and which further comprises (iii) a protein comprising the amino acid sequence of SEQ ID NO:19, or a protein comprising an amino acid sequence which has 95% or more homology to the amino acid sequence of SEQ ID NO:19 and having the ability to synthesize a dipeptide, or a protein encoded by a DNA that hybridizes with DNA encoding SEQ ID NO:19 at 65° C. in the presence of 0.7 to 1.0 mol/l sodium chloride followed by washing at 65° C. with a 0.1 to 2×SSC solution and encoding a protein having the activity to synthesize a dipeptide,
wherein said microorganism has the ability to produce the dipeptide, said ability to form said dipeptide being provided by said protein (iii); and
wherein said dipeptide is represented by formula (I):
R¹-R² (I)
(wherein R¹ is a free amino acid selected from the group consisting of L-alanine, L-methionine, L-serine, β-alanine, L-threonine, glycine, L-cysteine and L-α-aminobutyric acid, and R² is a free amino acid selected from the group consisting of L-alanine, L-glutamine, L-glutamic acid, glycine, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, β-alanine, L-azaserine, L-theanine, L-4-hydroxyproline, L-3-hydroxyproline, L-ornithine, L-citrulline and L-6-diazo-5-oxo-norleucine, except that R¹-R² cannot be L-alanyl-L-alanine).

23. The isolated microorganism according to claim 22, which has further lost activity normally provided by (i) at least one protein comprising the amino acid sequence of SEQ ID NOS:1, 2 and 4, or (ii) at least one protein that has a 95% or more homology to a protein comprising any one of the amino acid sequences of SEQ ID NOS:1, 2 and 4.

24. The isolated microorganism according to claim 22, wherein the dipeptide is Ala-Gln.

25. The isolated microorganism according to claim 23, wherein the dipeptide is Ala-Gln.

26. The microorganism according to claim 22, wherein the microorganism carries DNA that encodes said protein comprising the amino acid sequence of SEQ ID NO:19.

27. The microorganism according to claim 22, wherein the microorganism carries DNA that encodes said protein comprising an amino acid sequence which has 95% or more homology to the amino acid sequence of SEQ ID NO:19 and having the ability to synthesize a dipeptide.

28. The microorganism according to claim 22, wherein the microorganism carries said DNA that hybridizes with DNA encoding SEQ ID NO:19.

29. The microorganism according to claim 23, wherein the microorganism carries DNA that encodes said protein comprising the amino acid sequence of SEQ ID NO:19.

30. The microorganism according to claim 23, wherein the microorganism carries DNA that encodes said protein comprising an amino acid sequence which has 95% or more homology to the amino acid sequence of SEQ ID NO:19 and having the ability to synthesize a dipeptide.

31. The microorganism according to claim 23, wherein the microorganism carries said DNA that hybridizes with DNA encoding SEQ ID NO:19.

32. The microorganism according to claim 26, wherein said DNA is recombinant DNA ligated to a vector DNA.

33. The microorganism according to claim 27, wherein said DNA is recombinant DNA ligated to a vector DNA.

34. The microorganism according to claim 28, wherein said DNA is recombinant DNA ligated to a vector DNA.

35. The microorganism according to claim 29, wherein said DNA is recombinant DNA ligated to a vector DNA.

36. The microorganism according to claim 30, wherein said DNA is recombinant DNA ligated to a vector DNA.

37. The microorganism according to claim 31, wherein said DNA is recombinant DNA ligated to a vector DNA.


SEQUENCE LISTING (72 sequences)

SEQ ID NO: 1 - protein, 503 aa, Escherichia coli
MEFSVKSGSPEKQRSACIVVGVFEPRRLSPIAEQLDKISDGYISALLRRGELEGKPGQTLLLHHVPNVLSERILLIGCGKERELDERQYKQVIQKTINTLNDTGSMEAVCFLTELHVKGRNNYWKVRQAVETAKETLYSFDQLKTNKSEPRRPLRKMVFNVPTRRELTSGERAIQHGLAIAAGIKAAKDLGNMPPNICNAAYLASQARQLADSYSKNVITRVIGEQQMKELGMHSYLAVGQGSQNESLMSVIEYKGNASEDARPIVLVGKGLTFDSGGISIKPSEGMDEMKYDMCGAAAVYGVMRMVAELQLPINVIGVLAGCENMPGGRAYRPGDVLTTMSGQTVEVLNTDAEGRLVLCDVLTYVERFEPEAVIDVATLTGACVIALGHHITGLMANHNPLAHELIAASEQSGDRAWRLPLGDEYQEQLESNFADMANIGGRPGGAITAGCFLSRFTRKYNWAHLDIAGTAWRSGKAKGATGRPVALLAQFLLNRAGFNGEE

SEQ ID NO: 2 - protein, 427 aa, Escherichia coli
MTEAMKITLSTQPADARWGEKATYSINNDGITLHLNGADDLGLIQRAARKIDGLGIKHVQLSGEGWDADRCWAFWQGYKAPKGTRKVVWPDLDDAQRQELDNRLMIIDWVRDTINAPAEELGPSQLAQRAVDLISNVAGDRVTYRITKGEDLREQGYMGLHTVGRGSERSPVLLALDYNPTGDKEAPVYACLVGKGITFDSGGYSIKQTAFMDSMKSDMGGAATVTGALAFAITRGLNKRVKLFLCCADNLISGNAFKLGDIITYRNGKKVEVMNTDAEGRLVLADGLIDASAQKPEMIIDAATLTGAAKTALGNDYHALFSFDDALAGRLLASAAQENEPFWRLPLAEFHRSQLPSNFAELNNTGSAAYPAGASTAAGFLSHFVENYQQGWLHIDCSATYRKAPVEQWSAGATGLGVRTIANLLTA

SEQ ID NO: 3 - protein, 485 aa, Escherichia coli
MSELSQLSPQPLWDIFAKICSIPHPSYHEEQLAEYIVGWAKEKGFHVERDQVGNILIRKPATAGMENRKPVVLQAHLDMVPQKNNDTVHDFTKDPIQPYIDGEWVKARGTTLGADNGIGMASALAVLADENVVHGPLEVLLTMTEEAGMDGAFGLQGNWLQADILINTDSEEEGEIYMGCAGGIDFTSNLHLDREAVPAGFETFKLTLKGLKGGHSGGEIHVGLGNANKLLVRFLAGHAEELDLRLIDFNGGTLRNAIPREAFATIAVAADKVDVLKSLVNTYQEILKNELAEKEKNLALLLDSVANDKAALIAKSRDTFIRLLNATPNGVIRNSDVAKGVVETSLNVGVVTMTDNNVEIHCLIRSLIDSGKDYVVSMLDSLGKLAGAKTEAKGAYPGWQPDANSPVMHLVRETYQRLFNKTPNIQIIHAGLECGLFKKPYPEMDMVSIGPTITGPHSPDEQVHIESVGHYWTLLTELLKEIPAK

SEQ ID NO: 4 - protein, 870 aa, Escherichia coli
MTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 5 - protein, 535 aa, Escherichia coli
MRISLKKSGMLKLGLSLVAMTVAASVQAKTLVYCSEGSPEGFNPQLFTSGTTYDASSVPLYNRLVEFKIGTTEVIPGLAEKWEVSEDGKTYTFHLRKGVKWHDNKEFKPTRELNADDVVFSFDRQKNAQNPYHKVSGGSYEYFEGMGLPELISEVKKVDDNTVQFVLTRPEAPFLADLAMDFASILSKEYADAMMKAGTPEKLDLNPIGTGPFQLQQYQKDSRIRYKAFDGYWGTKPQIDTLVFSITPDASVRYAKLQKNECQVMPYPNPADIARMKQDKSINLMEMPGLNVGYLSYNVQKKPLDDVKVRQALTYAVNKDAIIKAVYQGAGVSAKNLIPPTMWGYNDDVQDYTYDPEKAKALLKEAGLEKGFSIDLWAMPVQRPYNPNARRMAEMIQADWAKVGVQAKIVTYEWGEYLKRAKDGEHQTVMMGWTGDNGDPDNFFATLFSCAASEQGSNYSKWCYKPFEDLIQPARATDDHNKRVELYKQAQVVMHDQAPALIIAHSTVFEPVRKEVKGYVVDPLGKHHFENVSIE

SEQ ID NO: 6 - protein, 339 aa, Escherichia coli
MLQFILRRLGLVIPTFIGITLLTFAFVHMIPGDPVMIMAGERGISPERHAQLLAELGLDKPMWQQYLHYIWGVMHGDLGISMKSRIPVWEEFVPRFQATLELGVCAMIFATAVGIPVGVLAAVKRGSIFDHTAVGLALTGYSMPIFWWGMMLIMLVSVHWNLTPVSGRVSDMVFLDDSNPLTGFMLIDTAIWGEDGNFIDAVAHMILPAIVLGTIPLAVIVRMTRSSMLEVLGEDYIRTARAKGLTRMRVIIVHALRNAMLPVVTVIGLQVGTLLAGAILTETIFSWPGLGRWLIDALQRRDYPVVQGGVLLVATMIILVNLLVDLLYGVVNPRIRHKK

SEQ ID NO: 7 - protein, 300 aa, Escherichia coli
MSQVTENKVISAPVPMTPLQEFWHYFKRNKGAVVGLVYVVIVLFIAIFANWIAPYNPAEQFRDALLAPPAWQEGGSMAHLLGTDDVGRDVLSRLMYGARLSLLVGCLVVVLSLIMGVILGLIAGYFGGLVDNIIMRVVDIMLALPSLLLALVLVAIFGPSIGNAALALTFVALPHYVRLTRAAVLVEVNRDYVTASRVAGAGAMRQMFINIFPNCLAPLIVQASLGFSNAILDMAALGFLGMGAQPPTPEWGTMLSDVLQFAQSAWWVVTFPGLAILLTVLAFNLMGDGLRDALDPKLKQ

SEQ ID NO: 8 - protein, 327 aa, Escherichia coli
MALLNVDKLSVHFGDESAPFRAVDRISYSVKQGEVVGIVGESGSGKSVSSLAIMGLIDYPGRVMAEKLEFNGQDLQRISEKERRNLVGAEVAMIFQDPMTSLNPCYTVGFQIMEAIKVHQGGNKSTRRQRAIDLLNQVGIPDPASRLDVYPHQLSGGMSQRVMIAMAIACRPKLLIADEPTTALDVTIQAQIIELLLELQQKENMALVLITHDLALVAEAAHKIIVMYAGQVVETGDAHAIFHAPRHPYTQALLRALPEFAQDKERLASLPGVVPGKYDRPNGCLLNPRCPYATDRCRAEEPALNMLADGRQSKCHYPLDDAGRPTL

SEQ ID NO: 9 - protein, 334 aa, Escherichia coli
MSTQEATLQQPLLQAIDLKKHYPVKKGMFAPERLVKALDGVSFNLERGKTLAVVGESGCGKSTLGRLLTMIEMPTGGELYYQGQDLLKHDPQAQKLRRQKIQIVFQNPYGSLNPRKKVGQILEEPLLINTSLSKEQRREKALSMMAKVGLKTEHYDRYPHMFSGGQRQRIAIARGLMLDPDVVIADEPVSALDVSVRAQVLNLMMDLQQELGLSYVFISHDLSVVEHIADEVMVMYLGRCVEKGTKDQIFNNPRHPYTQALLSATPRLNPDDRRERIKLSGELPSPLNPPPGCAFNARCRRRFGPCTQLQPQLKDYGGQLVACFAVDQDENPQR

SEQ ID NO: 10 - DNA, 1509 bp, Escherichia coli
atggagtttagtgtaaaaagcggtagcccggagaaacagcggagtgcctgcatcgtcgtgggcgtcttcgaaccacgtcgcctttctccgattgcagaacagctcgataaaatcagcgatgggtacatcagcgccctgctacgtcggggcgaactggaaggaaaaccggggcagacattgttgctgcaccatgttccgaatgtactttccgagcgaattctccttattggttgcggcaaagaacgtgagctggatgagcgtcagtacaagcaggttattcagaaaaccattaatacgctgaatgatactggctcaatggaagcggtctgctttctgactgagctgcacgttaaaggccgtaacaactactggaaagtgcgtcaggctgtcgagacggcaaaagagacgctctacagtttcgatcagctgaaaacgaacaagagcgaaccgcgtcgtccgctgcgtaagatggtgttcaacgtgccgacccgccgtgaactgaccagcggtgagcgcgcgatccagcacggtctggcgattgccgccgggattaaagcagcaaaagatctcggcaatatgccgccgaatatctgtaacgccgcttacctcgcttcacaagcgcgccagctggctgacagctacagcaagaatgtcatcacccgcgttatcggcgaacagcagatgaaagagctggggatgcattcctatctggcggtcggtcagggttcgcaaaacgaatcgctgatgtcggtgattgagtacaaaggcaacgcgtcggaagatgcacgcccaatcgtgctggtgggtaaaggtttaaccttcgactccggcggtatctcgatcaagccttcagaaggcatggatgagatgaagtacgatatgtgcggtgcggcagcggtttacggcgtgatgcggatggtcgcggagctacaactgccgattaacgttatcggcgtgttggcaggctgcgaaaacatgcctggcggacgagcctatcgtccgggcgatgtgttaaccaccatgtccggtcaaaccgttgaagtgctgaacaccgacgctgaaggccgcctggtactgtgcgacgtgttaacttacgttgagcgttttgagccggaagcggtgattgacgtggcgacgctgaccggtgcctgcgtgatcgcgctgggtcatcatattactggtctgatggcgaaccataatccgctggcccatgaactgattgccgcgtctgaacaatccggtgaccgcgcatggcgcttaccgctgggtgacgagtatcaggaacaactggagtccaattttgccgatatggcgaacattggcggtcgtcctggtggggcgattaccgcaggttgcttcctgtcacgctttacccgtaagtacaactgggcgcacctggatatcgccggtaccgcctggcgttctggtaaagcaaaaggcgccaccggtcgtccggtagcgttgctggcacagttcctgttaaaccgcgctgggtttaacggcgaagag

SEQ ID NO: 11 - DNA, 1281 bp, Escherichia coli
atgacagaagcgatgaagattaccctctctacccaacctgccgacgcgcgctggggagaaaaagcaacttacagcattaataatgacggcattaccctgcatttgaacggggcagacgatctggggctgatccagcgtgcggcgcgcaagattgacggtctgggcatcaagcatgttcagttaagcggtgaaggctgggatgcggatcgctgctgggcattctggcaaggttacaaagccccgaaaggcacgcgtaaagtggtgtggccggatctggacgatgcccagcgccaggaactggataaccgcctgatgatcatcgactgggtgcgtgacaccatcaacgcaccggcagaagaattgggaccatcgcaactggcacagcgtgctgttgatctgatcagcaacgtcgcgggcgatcgtgtgacttatcggatcaccaaaggcgaagatctgcgtgagcaaggttatatggggctgcacacagtcggacgcggttcagaacgttctccggtattgctggcgctggattacaacccaactggcgataaagaagcgccagtgtacgcgtgcctggtaggtaaaggtatcacttttgactccggcggctacagcatcaaacagactgcgtttatggactcgatgaagtcggacatgggcggcgcggcaacggttaccggggcgctggcatttgccattacgcgcggactgaacaagcgcgtgaagctgttcctctgctgtgcggataacctgattagcggcaatgcgttcaagctgggcgatatcatcacctatcgcaacggtaaaaaagttgaagtgatgaacactgatgccgaagggcgtctggtgcttgccgatggtctgattgatgccagtgcgcagaaaccggaaatgatcattgatgcggcgaccctcaccggggcggcgaaaactgcgctgggtaatgattatcacgcgctgttcagttttgacgatgcgctggccggtcgcttgctggcgagtgccgcgcaggagaacgaaccgttctggcgtctgccgctggcggagttccaccgcagccagctgccgtctaactttgccgaactgaacaataccggaagcgcggcgtatccggcaggcgcgagcacggcggcgggcttcctgtcgcactttgttgagaactatcagcaaggctggctgcatatcgactgctcggcgacttaccgtaaagcgccggttgaacagtggtctgcgggcgctacgggacttggtgtgcgcacgatagctaatctgttaacggcg

SEQ ID NO: 12 - DNA, 1455 bp, Escherichia coli
gtgtctgaactgtctcaattatctccacagccgctgtgggatatttttgccaaaatctgttctattcctcacccgtcctatcatgaagagcaactcgctgaatacattgttggttgggcaaaagagaaaggtttccatgtcgaacgcgatcaggtaggtaatatcctgattcgtaaacctgctaccgcaggtatggaaaatcgtaaaccggtcgtcttacaggcccacctcgatatggtgccgcagaaaaataacgacaccgtgcatgacttcacgaaagatcctatccagccttatattgatggcgaatgggttaaagcgcgcggcaccacgctgggtgcggataacggcattggtatggcctctgcgctggcggttctggctgacgaaaacgtggttcacggcccgctggaagtgctgctgaccatgaccgaagaagccggtatggacggtgcgttcggcttacagggcaactggttgcaggctgatattctgattaacaccgactccgaagaagaaggtgaaatctacatgggttgtgcggggggtatcgacttcacctccaacctgcatttagatcgtgaagcggttccagctggttttgaaaccttcaagttaaccttaaaaggtctgaaaggcggtcactccggcggggaaatccacgttgggctgggtaatgccaacaaactgctggtgcgcttcctggcgggtcatgcggaagaactggatctgcgccttatcgatttcaacggcggcacactgcgtaacgccatcccgcgtgaagcctttgcgaccattgctgtcgcagctgataaagtcgacgtcctgaaatctctggtgaatacctatcaggagatcctgaaaaacgagctggcagaaaaagagaaaaatctggccttgttgctggactctgtagcgaacgataaagctgccctgattgcgaaatctcgcgatacctttattcgtctgctgaacgccaccccgaacggtgtgattcgtaactccgatgtagccaaaggtgtggttgaaacctccctgaacgtcggtgtggtgaccatgactgacaataacgtagaaattcactgcctgatccgttcactgatcgacagcggtaaagactacgtggtgagcatgctggattcgctgggtaaactggctggcgcgaaaaccgaagcgaaaggcgcatatcctggctggcagccggacgctaattctccggtgatgcatctggtacgtgaaacctatcagcgcctgttcaacaagacgccgaacatccagattatccacgcgggcctggaatgtggtctgttcaaaaaaccgtatccggaaatggacatggtttctatcgggccaactatcaccggtccacactctccggatgagcaagttcacatcgaaagcgtaggtcattactggacactgctgactgaactgctgaaagaaattccggcgaag

SEQ ID NO: 13 - DNA, 2610 bp, Escherichia coli
atgactcaacagccacaagccaaataccgtcacgattatcgtgcgccggattaccagattactgatattgacttgacctttgacctcgacgcgcaaaagacggtcgttaccgcggtcagccaggctgtccgtcatggtgcatcagatgctccccttcgtctcaacggcgaagacctcaaactggtttctgttcatattaatgatgagccgtggaccgcctggaaagaagaagagggcgcactggttatcagtaatttgccggagcgttttacgcttaagatcattaatgaaataagcccggcggcgaataccgcgctggaagggctttatcagtcaggcgatgcgctttgcacccagtgtgaagccgaaggtttccgccatattacgtattatctcgaccgcccggacgtgctggcgcgttttaccaccaaaattattgccgataaaatcaaatatcccttcctgctttccaatggtaaccgcgttgcgcaaggcgaactggaaaacggacgccattgggtacagtggcaggacccgttcccgaaaccgtgctacctgtttgcgctggtggcaggcgactttgatgtactgcgcgatacctttaccacgcgttctggtcgcgaagtagcactggagctgtacgtcgatcgcggcaaccttgatcgcgcgccgtgggcgatgacctcgctgaaaaactccatgaaatgggatgaagaacgctttggcctggagtatgacctcgacatctatatgatcgtcgcggtggatttcttcaatatgggcgcaatggagaataaggggctgaatatctttaactccaaatatgtgctggcccgcaccgacaccgccaccgacaaagattacctcgatattgaacgcgttatcggccatgaatatttccataactggaccggtaaccgagtgacctgtcgcgactggttccagctcagcctgaaagaaggtttaaccgtcttccgcgatcaggagttcagctctgaccttggttcccgcgcagttaaccgcatcaataatgtacgcaccatgcgcggattgcagtttgcagaagacgccagcccgatggcgcacccgatccgcccggatatggtcattgagatgaacaacttctacaccctgaccgtttacgagaagggcgcggaagtgattcgcatgatccacaccctgcttggcgaagaaaacttccagaaagggatgcagctttatttcgagcgtcatgatggtagtgcagcgacctgtgacgactttgtgcaggcgatggaagatgcgtcgaatgtcgatctctcccatttccgccgttggtacagccagtccggtacaccgattgtgaccgtcaaagacgactacaatccggaaaccgagcagtacaccctgaccatcagccagcgcacgccagccacgccggatcaggcagaaaaacagccgctgcatattccgtttgccatcgaactgtatgataacgaaggcaaagtgatcccgttgcagaaaggcggtcatccggtgaattccgtgctgaacgtcactcaggcggaacagacctttgtctttgataatgtctacttccagccggtgcctgcgctgctgtgcgaattctctgcgccagtgaaactggaatataagtggagcgatcagcaactgaccttcctgatgcgtcatgcgcgtaatgatttctcccgctgggatgcggcgcaaagtttgctggcaacctacatcaagctgaacgtcgcgcgtcatcagcaaggtcagccgctgtctctgccggtgcatgtggctgatgctttccgcgcggtactgcttgatgagaagattgatccagcgctggcggcagaaatcctgacgctgccttctgtcaatgaaatggctgaattgttcgatatcatcgacccgattgctattgccgaagtacgcgaagcactcactcgtactctggcgactgaactggcggatgagctactggctatttacaacgcgaattaccagagcgagtaccgtgttgagcatgaagatattgcaaaacgcactctgcgtaatgcctgcctgcgtttcctcgcttttggtgaaacgcatctggctgatgtgctggtgagcaagcagttccacgaagcaaacaatatgactgatgcgctggcggcgctttctgcggcggttgccgcacagctgccttgccgtgacgcgctgatgcaggagtacgacgacaagtggcatcagaacggtctggtgatggataaatggtttatcctgcaagccaccagcccggcggcgaatgtgctggagacggtgcgcggcctgttgcagcatcgctcatttaccatgagcaacccgaaccgtattcgttcgttgattggcgcgtttgcgggcagcaatccggcagcgttccatgccgaagatggcagcggttacctgttcctggtggaaatgcttaccgacctcaacagccgtaacccgcaggtggcttcacgtctgattgaaccgctgattcgcctgaaacgttacgatgccaaacgtcaggagaaaatgcgcgcggcgctggaacagttgaaagggctggaaaatctctctggcgatctgtacgagaagataactaaagcactggct

SEQ ID NO: 14 - DNA, 1605 bp, Escherichia coli
atgcgtatttccttgaaaaagtcagggatgctgaagcttggtctcagcctggtggctatgaccgtcgcagcaagtgttcaggctaaaactctggtttattgctcagaaggatctccggaagggtttaacccgcagctgtttacctccggcaccacctatgacgcctcttccgtcccgctttataaccgtctggttgaatttaaaatcggcaccaccgaagtgatcccgggcctcgctgaaaagtgggaagtcagcgaagacggtaaaacctataccttccatctgcgtaaaggtgtgaagtggcacgacaataaagaattcaaaccgacgcgtgaactgaacgccgatgatgtggtgttctcgttcgatcgtcagaaaaacgcgcaaaacccgtaccataaagtttctggcggcagctacgaatacttcgaaggcatgggcttgccagagctgatcagtgaagtgaaaaaggtggacgacaacaccgttcagtttgtgctgactcgcccggaagcgccgttcctcgctgacctggcaatggacttcgcctctattctgtcaaaagaatatgctgatgcgatgatgaaagccggtacaccggaaaaactggacctcaacccaatcggaaccggtccgttccagttacagcagtatcaaaaagattcccgtatccgctacaaagcgtttgatggctactggggcaccaaaccgcagatcgatacgctggttttctctattacccctgacgcttccgtgcgttacgcgaaattgcagaagaatgaatgccaggtgatgccgtacccgaacccggcagatatcgctcgcatgaagcaggataaatccatcaatctgatggaaatgccggggctgaacgtcggttatctctcgtataacgtgcagaaaaaaccactcgatgacgtgaaagttcgccaggctctgacctacgcggtgaacaaagacgcgatcatcaaagcggtttatcagggcgcgggcgtatcagcgaaaaacctgatcccgccaaccatgtggggctataacgacgacgttcaggactacacctacgatcctgaaaaagcgaaagccttgctgaaagaagcgggtctggaaaaaggtttctccatcgacctgtgggcgatgccggtacaacgtccgtataacccgaacgctcgccgcatggcggagatgattcaggcagactgggcgaaagtcggcgtgcaggccaaaattgtcacctacgaatggggtgagtacctcaagcgtgcgaaagatggcgagcaccagacggtaatgatgggctggactggcgataacggggatccggataacttcttcgccaccctgttcagctgcgccgcctctgaacaaggctccaactactcaaaatggtgctacaaaccgtttgaagatctgattcaaccggcgcgtgctaccgacgaccacaataaacgcgttgaactgtacaaacaagcgcaggtggtgatgcacgatcaggctccggcactgatcatcgctcactccaccgtgtttgaaccggtacgtaaagaagttaaaggctatgtggttgatccattaggcaaacatcacttcgaaaacgtctctatcgaa

SEQ ID NO: 15 - DNA, 1017 bp, Escherichia coli
atgttgcagtttattctccgacgtttgggactcgtcatccccacgtttatcggtattacccttctcacatttgcctttgtccacatgatcccgggcgatccggtgatgatcatggcgggcgaacgtgggatctccccagagcgtcacgcgcagctgctggctgaactcggcttagataaaccgatgtggcagcagtatctccattacatttggggcgttatgcatggcgatctaggcatttcaatgaaaagccgcatcccggtttgggaagagttcgtgccgcgcttccaggccacgctggaacttggcgtctgcgcgatgatttttgctacggcagtcggtattccggtcggcgtgctggctgcggttaaacgcggttccattttcgatcacacagcggttggcctggcgctgacaggttattcaatgcctatcttctggtggggcatgatgctgatcatgctggtttcggtgcactggaacctgacgcccgtctccggtcgcgtgagcgatatggtgttcctcgatgactccaatccgttaaccggttttatgctaatcgacaccgccatctggggtgaagacggcaactttatcgatgccgtcgcccatatgatcttgcctgccattgtgctgggtactattccgctggcggtcattgtgcgtatgacacgctcctcgatgctggaagtgctgggcgaggattacatccgcaccgcgcgcgccaaagggctaacccgcatgcgggtgattatcgtccatgcgctgcgtaacgcgatgctgccggtggtgaccgttatcggcctgcaggtgggaacattgctggcgggggcgattctgaccgaaaccatcttctcgtggcccggtctgggacgctggttgattgacgcactgcaacgccgcgactatccggtagtgcagggcggcgtattgctggtggcgacgatgattatcctcgtcaacttgctggtcgatctgctgtacggcgtggtgaacccgcgtattcgtcataagaag

SEQ ID NO: 16 - DNA, 900 bp, Escherichia coli
atgtcacaggttactgaaaataaagtgattagcgcaccggtgccgatgaccccgttacaggagttctggcactattttaaacgcaacaaaggcgcggtcgtcgggctggtttacgtcgtcatcgtgctgttcatcgcgatctttgccaactggattgcaccctataacccggcggaacagttccgcgatgcactgctcgccccgccagcctggcaggaaggcggcagcatggcgcacttgctgggcaccgatgacgtaggccgtgatgtgctgtcgcgcctgatgtacggtgcgcgcctgtcgctgctggttggctgtctggtagttgtgttatcgctgattatgggcgttattctcggcctgatcgccggttactttggcggcctggtcgataacatcattatgcgcgtggtcgatatcatgctggcgctgccaagtctgctgctggcgctggtgctggtggcaattttcggcccgtcgattggtaacgccgcgctggcactgaccttcgttgccttgccgcactatgtgcgcttaacccgcgccgccgtgctggtggaagttaaccgcgattacgtcaccgcgtctcgcgtggcgggtgccggggcgatgcgtcagatgtttattaacatcttcccgaactgccttgcgccgctgattgttcaggcgtcgctcggtttctctaacgccattctcgatatggctgctcttggtttcctcggcatgggggcacagccgccaacgcctgagtggggcaccatgctctccgacgtgttgcagttcgcgcaaagcgcctggtgggtcgtgaccttcccgggtctggcgatcctgctgacggtgctggcatttaacctgatgggtgacggtctgcgtgacgcgctcgatcccaaactgaagcag

SEQ ID NO: 17 - DNA, 981 bp, Escherichia coli
atggcgttattaaatgtagataaattatcggtgcatttcggcgacgaaagcgcgccgttccgcgccgtagaccgcatcagctacagcgtaaaacagggcgaagtggtcgggattgtgggtgagtccggctccggtaagtcggtcagttcactggcgattatggggctgattgattatccgggccgcgtaatggcagaaaaactggagtttaacggccaggatttgcagcgtatctcagaaaaagagcgccgcaacctggtgggtgccgaagtggcgatgatcttccaggacccgatgaccagccttaacccgtgctacaccgtgggtttccagattatggaagcgattaaggtgcatcagggcggcaacaaaagtacccgccgtcagcgagcgattgacctgctgaatcaggtcggtattcccgatccggcatcgcgtctggatgtttacccgcatcagctttccggcggcatgagccagcgcgtgatgatcgccatggcgattgcctgtcggccaaaactgctgattgccgatgaaccgaccaccgcgctggacgtgaccattcaggcgcaaatcatcgaactactgctggagctacagcagaaagagaacatggcgctggtgttaattacccatgacctggcgctggtggcggaagcggcacataaaatcatcgtgatgtatgcaggccaggtggtggaaaccggtgatgcgcacgccatcttccatgcgccgcgtcacccgtatactcaggcattgctgcgtgcgctgccagaatttgctcaggacaaagaacgtctggcgtcgttgccaggtgtcgttcccggcaagtacgaccgcccgaacggctgcctgcttaacccgcgctgcccctatgccactgacagatgtcgcgctgaagaaccggcgctgaatatgctcgctgacgggcgtcagtccaaatgccattacccacttgatgatgccgggaggccgacacta

SEQ ID NO: 18 - DNA, 1002 bp, Escherichia coli
atgagtacgcaagaggccaccctgcaacaaccgctgttgcaggctatcgacctgaaaaaacattatccggtgaagaaaggcatgttcgcgccggaacgtctggttaaagcgctggatggcgtttcgtttaaccttgaacgtggcaaaacgctggcagtagtgggcgaatctggctgcggtaaatcgaccctcggtcggttgctgacgatgattgaaatgcccaccggtggcgagctgtattaccaggggcaggatctgcttaagcacgatccgcaggcgcagaagctgcgtcggcagaaaatccagatcgtcttccagaacccttacggttcgctgaatccgcgtaaaaaagtcgggcaaattcttgaagagccgctgctgatcaacaccagcttaagcaaagaacagcgtcgggaaaaagccctgtcgatgatggcgaaagtcggcctgaaaaccgagcactatgaccgctatccgcatatgttctccggcggtcagcgtcagcgtatcgccatcgcccgtggtctgatgctcgacccggatgtggtgattgccgatgaaccggtttccgcgctggatgtttcagtgcgcgcgcaggtgctgaatctgatgatggatttgcagcaggagttggggctgtcttatgtctttatctcccacgacctgtcggtggtggagcacattgctgatgaagtgatggtgatgtacctgggccgctgcgtggagaagggaacgaaagaccaaatcttcaataacccgcgccatccgtacactcaggcgctactttccgcgacgccgcgcctgaacccggacgatcgccgcgagcgcatcaagctcagcggtgaactaccaagcccactgaatccaccgccgggttgcgccttcaacgcccgctgtcgtcggcgcttcggcccctgcacccagttgcagccgcagctaaaagactacggcggtcaactggtagcttgttttgctgttgatcaggatgaaaatccgcagcgt

SEQ ID NO: 19 - protein, 472 aa, Bacillus subtilis
  Strain 168
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 20 - protein, 472 aa, Bacillus subtilis
  ATCC6633
MERKTVLVIADLGGCPPHMFYKSAAEKYNLVSFIPRPFAITASHAALIEKYSVAVIKDKDYFQSLADFEHPDSIYWAHEDHDKPEEEVVEQIVKVAQMFEADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVTFSAAAPGTSVDLTLFEAFNSIAAFELKGSNSQDVAESIRQIQQHAKLTAKYVLPV

SEQ ID NO: 21 - protein, 472 aa, Bacillus subtilis
  Strain IAM1213
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDLPDGLLKGDTEIVSFSAAAPGTSVDLTLFEAFNSIAAFELKGSNSQDVAESIRQIQQHAKLTAKYVLPV

SEQ ID NO: 22 - protein, 472 aa, Bacillus subtilis
  Strain IAM1107
MERKTVLVIADLGGCPPHMFYKSAAEKYNLVSFIPRPFAITASHAALIEKYSVAVVKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEVKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIFSFSAAAPGTSVDLTLFEAFNSIAAFELKGSNSQDVAESIRQIQQHAKLTAKYVLPV

SEQ ID NO: 23 - protein, 472 aa, Bacillus subtilis; identical to SEQ ID NO: 20
  Strain IAM1214

SEQ ID NO: 24 - protein, 472 aa, Bacillus subtilis
  ATCC21555
MERKTVLVIADLGGCPPHMFYKSAAEKYNLVSFIPRPFAITASHAALIEKYSIAVIKDKDYFKSLADFEHPDSIYWAHEDHDKPEEEVVEEIVKVADMFGVDAITTNNELFIAPMAKACKRLGLRGAGVQAAENARDKNKMRAAFNRAGVKSIKNKRVTTLEDFRAALQEIGTPLILKPTYLASSIGVTLIKEMETAEAEFNRVNEYLKSINVPKAVTFEAPFIAEEFLQGEYDDWYETSGYSDYISIEGIMADGEYFPVAIHDKTPQIGFTETAHITPSILDDDAKRKIVEAAKKANEGLGLENCATHTEIKLMKNREAGLIESAARFAGWNMIPNIKKVFGVDMAQLLLDVLCYGKEADLPKGLLEQEPCYVADCHLYPQHFKENGQLPETVVDFVIESIEIPDGVLKGDTELVSFSAAEAGTSVDLRLFEAFNSIAAFELKGSNSNDVAESIKQIQQQAKLTAKYALSV

SEQ ID NO: 25 - protein, 472 aa, Bacillus amyloliquefaciens
  Strain IFO3022
MERKTVLVIADLGGCPPHMFYKSAAEKYNLVSFIPRPFAITASHAALIEKYSVAVIKDKDYFKSLADFEHPDSIYWAHEDHDKPEEEVVEEIVKVAGMFAVDAITTNNELFIAPMAKACERLGLRGAGVQAAENARDKNKMRAAFNRAGVKSIKNRRVTTLEDFRAALQEIGTPLILKPTYLASSIGVTLIKERETAEAEFNRVNEYLKSINVPKAVTFEAPFIAEEFLQGEYDDWYETSGYSDYISIEGIMADGEYFPVAIHDKTPQIGFTETSHITPSILDDDAKRKIVEAAKKANEGLGLENCATHTEIKLMKNREAGLIESAARFAGWNMIPNIKKVFGVDMAQLLLDVLCFGKEADLPKGLLEQEPCYVADCHLYPQHFKENGQLPETAVDFVIESIDIPDGVLKGDTEIVSFSAAEAGTSVDLRLFEAFNSIAAFELKGSNSGDVAESIKQIQQQAKLTAKYALPV

SEQ ID NO: 26 - DNA, 1416 bp, Bacillus subtilis
  Strain 168
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgaacaccctgattccatttattgggcgcatgaagatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggactgcaaaattgcgcaacacatacagagatcaagctaatgaaaaacagagaaccgggtttaatagagtcggcagccagatttgccggctggaatatgatccccaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccaaattcctgaaactgctgaggatttggtcattgaagcgatcgatattccggacgggcttttaaaaggggatactgaaatcgtttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 27 - DNA, 1416 bp, Bacillus subtilis
  ATCC6633
atggagagaaaaacagtattggtcatcgctgatcttggaggctgcccgccgcacatgttttataaaagcgctgctgaaaaatataacctggttagctttattccgagaccttttgcaataacagcctcccatgcagcactgattgaaaaatactcggtcgcggtcataaaagataaagactattttcagagcttagctgattttgagcatcccgattcaatttattgggcgcatgaggatcatgacaagcctgaagaagaggttgtcgagcaaatcgtcaaggttgcccaaatgtttgaggcggacgccatcacaacaaacaatgaattattcattgccccgatggcgaaagcctgtgaacgccttggcctgaggggcgccggagtgcaggcagcggaaaatgccagagataaaaataaaatgagggacgcttttaataaggcgggagtcaaatcgatcaaaaacaaacgagtcacaactcttgaggattttcgtgctgcacttgaagagatcggcacacctctaatcttaaagcctacatacttagcgagttcaatcggcgtaacgctgattaccgacacggagacggcagaagatgaatttaacagagtcaatgactacctgaaatcgattaacgtgccgaaggcggtcacatttgaagcaccgtttattgctgaggaatttttacagggtgagtacggagactggtatcaaacagaagggtactccgactatatcagcatagaaggcattatggcagatggtgagtattttccgatcgccattcatgacaaaacgccgcaaattggatttacagagacatcacatattacgccatccattctggatgaagaggcgaaaaagaaaattgtcgaagcggctaaaaaggcaaatgaagggcttggactgcaaaattgcgcaacacatacagaaatcaagctaatgaaaaacagagaaccgggtttaatagagtcggctgccagattcgcaggctggaatatgattcctaacattaaaaaggttttcggccttgatatggcgcaattattattagatgttctctgtttcggaaaagatgctgatctgccggacgggttattggatcaagagccttactatgttgctgactgccatctgtaccctcagcatttcaaacaaaatggccagatccctgaaactgccgaggatttggtaatcgaagcgatcgatattccggatgggcttttgaagggtgatacagaaatcgttactttttcggctgcggcaccaggaacatcagttgatttgacactgtttgaagccttcaactccattgctgcatttgaactgaaaggcagcaattcacaggatgtggctgaatcaatcagacaaattcagcagcatgcgaagctgacggcaaagtatgtgctgccagta

SEQ ID NO: 28 - DNA, 1416 bp, Bacillus subtilis
  Strain IAM1213
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgactccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactctcgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaaggtcttggcctgcaaaattgcgcaacacatacagagatcaagctaatgaaaaatagagaaccgggtttaattgaatcggcagccagattcgccggctggaatatgatccccaatattaaaaaggtctttggccttgatatggcgcaattattattagatgtcctttgtttcggaaaagacgccgatctgccggacggattattggatcaagagccttattatgttgccgactgccatttgtacccgcaacatttcaaacaaaatggccagattccagaaactgctgaggatttggtcattgaagcgatcgatctgcctgacgggcttttaaaaggggatactgagatcgtttctttttcggccgcagcaccaggaacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 29 - DNA, 1416 bp, Bacillus subtilis
  Strain IAM1107
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcgtaaaagataaagactattttaagagtttagctgattttgagcatcctgactccatttattgggcgcatgaggatcataacaagcctgaggaagaggtcgtcgagcaaatcgtcaaggttgccgaaatgttcggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagaggtcaagctaatgaaaaacagagaaccgggtttaattgaatcggcagccagatttgccggctggaatatgatccctaacattaaaaaggttttcggccttgatatggcgcaattattattagatgtcctctgtttcggaaaagatgccgatctgccggacggattattggatcaagagccttactatgtcgccgactgccatttgtacccgcagcatttcaaacaaaatggccagattccagaaaccgctgaggatttggtcattgaagcgatcgatattccggacgggcttttaaaaggggatactgaaatcttttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 30 - DNA, 1416 bp, Bacillus subtilis; identical to SEQ ID NO: 27
  Strain IAM1214

SEQ ID NO: 31 - DNA, 1416 bp, Bacillus subtilis
  ATCC21555
atggagagaaaaacagtattggttatcgctgatcttgggggctgcccgccgcatatgttttacaaaagcgcagccgaaaaatacaacctcgtcagctttattccgagaccctttgcaattacagcctctcatgcggccttaattgaaaaatactcgattgcggtcattaaagataaagactattttaagagtctggctgattttgaacatcccgattcgatttattgggctcatgaagatcatgacaaacctgaggaagaagtcgtcgaagaaatcgtgaaagtggccgacatgtttggggttgacgccattacgaccaacaatgaactgtttatcgctccgatggcaaaagcgtgtaaacgtctcggcctgcggggagcgggcgtacaggccgctgaaaacgccagagataaaaataaaatgagagccgccttcaaccgggccggcgtcaaatccatcaaaaacaaacgggtgacgaccctggaagatttccgcgccgcgcttcaggaaatcggaacgccgcttattctgaagcctacatatctggcaagctcgatcggcgtgacgcttattaaagagatggaaacggccgaagctgaattcaacagagtcaatgagtacttgaaatcgattaatgtaccgaaagcggtgacgtttgaagcgccgtttatcgcggaagaattcttgcagggcgagtatgatgactggtacgaaacaagcggttattccgactatatcagcatcgaaggcatcatggccgacggagaatacttccccgttgcgatccatgataaaacaccgcaaatcggattcacggagacagcgcatattacgccgtccatcctggatgatgacgccaagcggaaaatcgtcgaagctgccaagaaggcgaatgaaggactcggcctcgaaaactgtgcaacgcatacagaaataaaattaatgaaaaaccgggaagccggactgattgagtcagcggccagattcgcgggatggaatatgattccgaatattaaaaaggtcttcggcgttgatatggcgcagctattattggatgttctctgttacggaaaagaagctgatctgccgaaaggattattggagcaggagccatgctatgtcgcagactgccacttgtatcctcagcatttcaaagagaacggccagctgcctgagacggttgtcgatttcgtcattgaaagcattgaaattcctgacggcgtcttaaagggagacactgaactcgtttctttctcagcggctgaggcgggtacgtcagtggatctgcggctgttcgaagcgttcaacagcattgcggcgtttgagctgaaaggaagcaattcgaacgacgtggccgaatcaatcaaacaaattcagcagcaggcgaagctgactgcaaagtatgcgttatcggta

SEQ ID NO: 32 - DNA, 1416 bp, Bacillus amyloliquefaciens
  Strain IFO3022
atggagagaaaaacagtattggttatcgctgaccttgggggatgcccgccgcatatgttttacaaaagcgcagccgaaaaatacaacctcgtcagctttattccgagaccttttgcaattacagcctctcatgcggcattaattgaaaaatactcggtcgcggtcataaaagataaagactattttaagagtctggctgattttgagcatcccgattcgatttactgggctcatgaagatcatgacaaacctgaggaagaagtagtcgaagaaatcgtcaaggtggccggcatgttcgcggttgacgccattacgaccaacaatgaactgtttatcgctccgatggcaaaagcgtgtgaacgtctcggcctgcggggagcgggcgtacaggccgctgaaaatgccagagataaaaacaaaatgagagccgctttcaaccgggccggcgtcaagtctatcaaaaacagacgggtgacgacgctggaagatttccgcgccgcgcttcaggaaatcggaacgccgctcattctgaagcctacatatctggcgagctccatcggcgtgacgctcatcaaagagagggaaacggccgaagccgaatttaacagagtcaatgaatacctgaagtcgatcaacgtaccgaaagcggtcacgtttgaagcgccgtttatcgcggaagaatttttgcagggcgagtatgacgactggtacgaaacaagcggttattccgactatatcagcatagaaggcatcatggccgacggagaatacttccctgtcgcaattcatgataaaacaccgcaaatcggattcacggagacatcgcatattacgccgtccatcctggatgatgacgcgaagcggaaaatcgtcgaagcagccaaaaaggcgaatgaaggactcggcctcgaaaactgcgcaacccatacagagattaaattaatgaaaaaccgggaagccggactgattgaatcagcggcacgatttgcgggctggaacatgattccgaatattaaaaaggtcttcggcgtcgatatggcgcagctgttattggatgttctctgtttcggaaaagaagccgatctgccgaaaggattattggagcaggagccgtgctatgtcgccgactgccacttgtatcctcagcatttcaaagagaacggccagctgcctgagacggctgtcgatttcgtcattgaaagcattgacattcccgacggcgtcttaaagggagacaccgaaatcgtttctttctcggcggccgaggcgggtacatccgtggatctgcggctgttcgaagcgttcaacagcattgcggcgttcgagctgaaaggaagcaattcgggtgacgtggccgaatcaatcaaacaaattcagcagcaggcgaagctgactgcaaagtatgcgttaccggta

SEQ ID NO: 33 - protein, 93 aa, Bacillus subtilis
  Strain 168
GAGVQAAENARDKNKMRDAFNKAGVKSIKNKRVTTLEDFRAALEEIGTPLILKPTYLASSIGVTLITDTETAEDEFNRVNDYLKSINVPKAVT

SEQ ID NO: 34 - DNA, 279 bp, Bacillus subtilis
  Strain 168
ggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacg

SEQ ID NO: 35 - DNA, 27 bp, Artificial Sequence
  Synthetic DNA
ttaaccatggagagaaaaacagtattg

SEQ ID NO: 36 - DNA, 30 bp, Artificial Sequence
  Synthetic DNA
atatggatcctactggcagcacatactttg

SEQ ID NO: 37 - DNA, 21 bp, Artificial Sequence
  Synthetic DNA
caccgcagacggaggatacac

SEQ ID NO: 38 - DNA, 22 bp, Artificial Sequence
  Synthetic DNA
cggacgtcacccaataatcgtg

SEQ ID NO: 39 - DNA, 34 bp, Artificial Sequence
  Synthetic DNA
gaagttcctatactttctagagaataggaacttc

SEQ ID NO: 40 - DNA, 56 bp, Artificial Sequence
  Synthetic DNA
ctaaccctgtgacctgcaatactgttttgcgggtgagtgtaggctggagctgcttc

SEQ ID NO: 41 - DNA, 56 bp, Artificial Sequence
  Synthetic DNA
gaaactgccggaaggcgattaaacgccatccggcagcatatgaatatcctccttag

SEQ ID NO: 42 - DNA, 56 bp, Artificial Sequence
  Synthetic DNA
ttacgcaacaggaatagactgaacaccagactctatgtgtaggctggagctgcttc

SEQ ID NO: 43 - DNA, 56 bp, Artificial Sequence
  Synthetic DNA
agaaaacaggggtaaattccccgaatggcggcgctacatatgaatatcctccttag

SEQ ID NO: 44 - DNA, 56 bp, Artificial Sequence
  Synthetic DNA
atggagtttagtgtaaaaagcggtagcccggagaaagtgtaggctggagctgcttc

SEQ ID NO: 45 - DNA, 56 bp, Artificial Sequence
  Synthetic DNA
ttactcttcgccgttaaacccagcgcggtttaacagcatatgaatatcctccttag

SEQ ID NO: 46 - DNA, 56 bp, Artificial Sequence
  Synthetic DNA
atgacagaagcgatgaagattaccctctctacccaagtgtaggctggagctgcttc

SEQ ID NO: 47 - DNA, 56 bp, Artificial Sequence
  Synthetic DNA
ttacgccgttaacagattagctatcgtgcgcacacccatatgaatatcctccttag

SEQ ID NO: 48 - DNA, 56 bp, Artificial Sequence
  Synthetic DNA
gcatccccacctcataacgttgacccgaccgggcaagtgtaggctggagctgcttc

SEQ ID NO: 49 - DNA, 56 bp, Artificial Sequence
  Synthetic DNA
ctgtacggcattttgctatgcttgtcgccactgttgcatatgaatatcctccttag

SEQ ID NO: 50 - DNA, 21 bp, Artificial Sequence
  Synthetic DNA
gtgtctgaactgtctcaatta

SEQ ID NO: 51 - DNA, 21 bp, Artificial Sequence
  Synthetic DNA
cggaatttctttcagcagttc

SEQ ID NO: 52 - DNA, 21 bp, Artificial Sequence
  Synthetic DNA
atgactcaacagccacaagcc

SEQ ID NO: 53 - DNA, 21 bp, Artificial Sequence
  Synthetic DNA
tgctttagttatcttctcgta

SEQ ID NO: 54 - DNA, 21 bp, Artificial Sequence
  Synthetic DNA
agtgcctgcatcgtcgtgggc

SEQ ID NO: 55 - DNA, 21 bp, Artificial Sequence
  Synthetic DNA
ggcgccttttgctttaccaga

SEQ ID NO: 56 - DNA, 21 bp, Artificial Sequence
  Synthetic DNA
gacgcgcgctggggagaaaaa

SEQ ID NO: 57 - DNA, 21 bp, Artificial Sequence
  Synthetic DNA
cgtagcgcccgcagaccactg

SEQ ID NO: 58 - DNA, 21 bp, Artificial Sequence
  Synthetic DNA
atgcgtatttccttgaaaaag

SEQ ID NO: 59 - DNA, 21 bp, Artificial Sequence
  Synthetic DNA
ttattcgatagagacgttttc

SEQ ID NO: 60 - DNA, 29 bp, Artificial Sequence
  Synthetic DNA
tacactcgagattaaagaggagaaattaa

SEQ ID NO: 61 - DNA, 30 bp, Artificial Sequence
  Synthetic DNA
ttaggatcctcatactggcagcacatactt

SEQ ID NO: 62 - DNA, 24 bp, Artificial Sequence
  Synthetic DNA
caagaattctcatgtttgacagct

SEQ ID NO: 63 - DNA, 28 bp, Artificial Sequence
  Synthetic DNA
taactcgagattccctttttacgtgaac

SEQ ID NO: 64 - DNA, 1428 bp, Bacillus pumilus
  NRRL B-12025
gtgctttcattgagtaaaaaaactgtacttgtcattgctgacttaggagggtgcccgccccatatgttttatgaaagcgtggcggcatcataccatatcgtttcttatatcccaagaccctttgcgattacaaagggacatgccgagctaatcgaaaaatactccattgccgtcatcaaagaccgtgattattttgagacacacccttcttttgaacaccctgattctatttactgggcacatgatgattatccaaaatcagaagaagaagttgtggaagacttcattcgagtagcttcctttttcaaagcagatgcaatcacgaccaataatgaattattcattgcaccgatggcaaaggccgctgaacgtcttgggctacgaggtgccggtgtcaaggcagccgaaatggcgcgtgataaaagccaaatgagggctgcattcaatgcctctggcgtcaaagcggtgaaaactcagcctgtcacgactttatctgatttccaacaagccattgagtctatcggaacaccgctcattttaaagcctacatatttagccagttctattggcgtcaccttgtttcatgacaaagccggaagtgatgacttgtttttacaagtacaatcgtatttggaaaccataccagtcccagacgctgtcacgtatgaagcaccgtttgtcgctgaaacatatttagagggtgcttacgaagattggtatgaagacgaaggatatgctgattatgtcagtgtagaagggctggtcgtagagggcgaatatctcccttttgtcatacatgataaaacccctcaaatcggctttacagaaacggctcatatcactccgacgatcttagacaatgaagccaagcaaatcatcattgaagcagcaaggaaggcaaatgaagggctaggtcttgaacattgtgcaacccatacagaaatcaaactcatgaaaaatcgagaaactggactgatcgaggcagcggctcgattcgctggctggaatatgatcccgaatattaaaaaagtctttggcgtcgatatggcgaagctattgattgatgtattagttgatggtaaaaaggctgtactgccaaaacagctgctttctggacatacattttatgtagcggactgccacctgtaccctcagcattttaaagagagtgggcttatcccgcctgaagccacacatattaccattgatcatgtgtctattccgcaggaagcattcgttggagatactgcgattgtcagtcaatcattccctgccaaagggactattgtggatcttgaattatttgaagcttttaatggaatcgtatctcttgaattaaaaggatcatcctcacaagatgttgccgcgtccatccgcaacattcagaaacaggcaacgattcagttaatggatgaattagtgaaggga

SEQ ID NO: 65 - DNA, 1416 bp, Bacillus subtilis
  ATCC 15245 and IAM 1033
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 66 - DNA, 23 bp, Artificial Sequence
  Synthetic DNA
ccgatggcraaagcstgtraacg

SEQ ID NO: 67 - DNA, 21 bp, Artificial Sequence
  Synthetic DNA
cggcagatcrgcdtcttttcc

SEQ ID NO: 68 - protein, 476 aa, Bacillus pumilus
  NRRL B-12025
VLSLSKKTVLVIADLGGCPPHMFYESVAASYHIVSYIPRPFAITKGHAELIEKYSIAVIKDRDYFETHPSFEHPDSIYWAHDDYPKSEEEVVEDFIRVASFFKADAITTNNELFIAPMAKAAERLGLRGAGVKAAEMARDKSQMRAAFNASGVKAVKTQPVTTLSDFQQAIESIGTPLILKPTYLASSIGVTLFHDKAGSDDLFLQVQSYLETIPVPDAVTYEAPFVAETYLEGAYEDWYEDEGYADYVSVEGLVVEGEYLPFVIHDKTPQIGFTETAHITPTILDNEAKQIIIEAARKANEGLGLEHCATHTEIKLMKNRETGLIEAAARFAGWNMIPNIKKVFGVDMAKLLIDVLVDGKKAVLPKQLLSGHTFYVADCHLYPQHFKESGLIPPEATHITIDHVSIPQEAFVGDTAIVSQSFPAKGTIVDLELFEAFNGIVSLELKGSSSQDVAASIRNIQKQATIQLMDELVKG

SEQ ID NO: 69 - DNA, 44 bp, Artificial Sequence
  Synthetic DNA
catgccatggagaaaaaaactgtacttgtcattgctgacttagg

SEQ ID NO: 70 - DNA, 38 bp, Artificial Sequence
  Synthetic DNA
cgcggatcccttcactaattcatccattaactgaatcg

SEQ ID NO: 71 - DNA, 26 bp, Artificial Sequence
  Synthetic DNA
gctaggtcttgaacattgtgcaaccc

SEQ ID NO: 72 - DNA, 23 bp, Artificial Sequence
  Synthetic DNA
ggtgttccgatagactcaatggc